(19) United States
(12) Patent Application Publication
(10) Pub. No.: US 2010/0248307 A1
(43) Pub. Date: Sep. 30, 2010

(54) PEPTIDE PRODUCTION METHOD

(75) Inventors: Kuniki Kino, Chiba (JP); Toshinobu Arai, Kanagawa (JP); Masahiro Kokubo, Gunma (JP); Makoto Yagasaki, Yamaguchi (JP)

Correspondence Address: FITZPATRICK CELLA HARPER & SCINTO, 1290 Avenue of the Americas, NEW YORK, NY, 10104-3800, US

(73) Assignee: KYOWA HAKKO BIO CO., LTD., Chiyoda-ku, Tokyo (JP)

(21) Appl. No.: 12/675,420

(22) PCT Filed: Jun. 19, 2008
(86) PCT No.: PCT/JP2008/061207

§ 371 Date: Feb. 26, 2010

(30) Foreign Application Priority Data
Aug. 31, 2007 (JP) 2007-225766

(51) Int. Cl. C12P 21/06; C12N 9/50; C07H 21/04; C12N 1/21

(52) U.S. Cl. 435/69.1; 435/219; 536/23.2; 435/252.33

(57) ABSTRACT

According to the present invention, a protein having peptide-synthesizing activity, a DNA encoding the protein, a recombinant DNA containing the DNA, a transformant obtained via transformation with the recombinant DNA, a process for producing a protein having peptide-synthesizing activity using the transformant and the like, a process for producing a peptide using a protein having peptide-synthesizing activity, and a process for producing a peptide using as an enzyme source a culture or the like of a transformant or a microorganism producing a protein having peptide-synthesizing activity are provided.

### TECHNICAL FIELD

The present invention relates to a protein having peptide-synthesizing activity, a DNA encoding the protein, a recombinant DNA containing the DNA, a transformant obtained via transformation with the recombinant DNA, a process for producing a protein having peptide-synthesizing activity, a process for producing a peptide using a protein having peptide-synthesizing activity, and a process for producing a peptide using a microorganism or a transformant that produces a protein having peptide-synthesizing activity.

### BACKGROUND ART

Regarding peptide synthesis by an enzymatic method, a method using a reverse reaction of protease (see J. Biol. Chem., 119, 707-720 (1937)), methods using thermostable aminoacyl t-RNA synthetase (see JP Patent Publication (Kokai) No. 58-146539 A (1983); JP Patent Publication (Kokai) No. 58-209991 A (1983); JP Patent Publication (Kokai) No. 58-209992 A (1983); and JP Patent Publication (Kokai) No. 59-106298 A (1984)), and methods using non-ribosomal peptide synthetase (hereinafter, referred to as NRPS) (see Chem. Biol., 7, 373-384 (2000); FEBS Lett., 498, 42-45 (2001); U.S. Pat. No. 5,795,738; and U.S. Pat. No. 5,652,116) are known.

Proteins known to form peptides by peptide bond forming activity at an α-carboxyl group of an L-amino acid are basilicin synthetase, which is a dipeptide antibiotic from a microorganism belonging to the genus Bacillus (see International Publication WO 2004/058960 (pamphlet)), diketopiperazine synthetase from Streptomyces albulus (see International Publication WO 2005/103260 (pamphlet)), and a protein from Ralstonia solanacearum (see International Publication WO 2006/101023 (pamphlet)).

However, products obtained with the use of the above enzymes are only dipeptides. Hence, a new enzyme is required, which differs from such enzymes and is intended for synthesis of a peptide with chain length longer than that of a dipeptide.

Currently, a biological synthesis method using a DNA recombination method is employed for long-chain peptides having a length of 50 residues or more, but is not efficient for synthesizing a peptide shorter than such length. Also, a method for producing a peptide using a novel peptide synthetase from a bacterium belonging to the genus Empedobacter has also been reported (see International Publication WO 2004/011652 (pamphlet)). However, in this case, peptide bond formation requires the use of a derivatized product such as an esterified product or an amidated product as an amino acid component that is elongated at the N-terminus, and synthesis from free amino acids cannot be performed.

Bacillus subtilis ATCC6633 is known to produce peptide antimicrobials, rhizocticin A (L-Arg-L-2-amino-5-phosphono-3-cis-pentenoic acid, L-Arg-L-APPA), rhizocticin B (L-Val-L-Arg-L-APPA), rhizocticin C (L-Ile-L-Arg-L-APPA), and rhizocticin D (L-Leu-L-Arg-L-APPA) (see Arch. Microbiol., 153, 276-281 (1990)). However, the biosynthetic pathway thereof, proteins involved in the biosynthesis thereof, and genes involved in the biosynthesis thereof remain unknown.

Regarding Bacillus licheniformis BL02410 (GenBank Accession No. AAU21844), Herpetosiphon aurantiacus HaurDRAFT_{—}4222 (GenBank Accession No. EAU19320), Streptococcus pneumoniae spr0969 (GenBank Accession No. AAK99773), Chromobacterium violaceum CV0806 (GenBank Accession No. AAQ58482) and/or Bifidobacterium adolescentis BAD1200 (GenBank Accession No. YP_{—}910063), the amino acid sequences of the proteins and the nucleotide sequences of the genes encoding the proteins are known, but information concerning their functions does not exist.

### DISCLOSURE OF THE INVENTION

### Object to Be Attained by the Invention

An object of the present invention is to provide a protein having peptide-synthesizing activity, a DNA encoding the protein, a recombinant DNA containing the DNA, a transformant obtained via transformation with the recombinant DNA, a process for producing a protein having peptide-synthesizing activity using the transformant or the like, a process for producing a peptide using the protein having peptide-synthesizing activity, and a process for producing a peptide using a culture or the like of a transformant or a microorganism that produces the protein having peptide-synthesizing activity as an enzyme source.

### Means for Attaining the Object

The present invention relates to the following (1) to (19).
(1) A protein according to any one of the following [1] to [3]:
[1] a protein having the amino acid sequence shown by any one selected from the group consisting of SEQ ID NOS: 2, 4, 6, 8, 10, and 12;
[2] a protein consisting of an amino acid sequence that has a deletion, a substitution, or an addition of one or more amino acids in the amino acid sequence shown by any one selected from the group consisting of SEQ ID NOS: 2, 4, 6, 8, 10, and 12, and having peptide-synthesizing activity; and
[3] a protein consisting of an amino acid sequence that has 80% or more homology with the amino acid sequence shown by any one selected from the group consisting of SEQ ID NOS: 2, 4, 6, 8, 10, and 12, and having peptide-synthesizing activity. (2) A DNA according to any one of the following [1] to [3]:
[1] a DNA encoding the protein of (1) above;
[2] a DNA having the nucleotide sequence shown by any one selected from the group consisting of SEQ ID NOS: 1, 3, 5, 7, 9, and 11; and
[3] a DNA hybridizing under stringent conditions to a DNA consisting of a nucleotide sequence complementary to the nucleotide sequence shown by any one selected from the group consisting of SEQ ID NOS: 1, 3, 5, 7, 9, and 11, and encoding a protein having peptide-synthesizing activity.
(3) A recombinant DNA containing the DNA of (2) above.
(4) A transformant having the recombinant DNA of (3) above.
(5) The transformant of (4) above, which is obtained using a microorganism as a host.
(6) The transformant of (5) above, wherein the microorganism belongs to the genus Escherichia.
(7) A process for producing the protein of (1), which comprises culturing a microorganism which has an ability to produce the protein of (1) in a medium, allowing the protein to form and accumulate in a culture, and recovering the protein from the culture.
(8) The process of (7) above, wherein the microorganism belongs to the genus Bacillus, the genus Herpetosiphon, the genus Streptococcus, the genus Chromobacterium, or the genus Bifidobacterium.
(9) The process of (8) above, wherein the microorganism belonging to the genus Bacillus, the genus Herpetosiphon, the genus Streptococcus, the genus Chromobacterium, or the genus Bifidobacterium belongs to species selected from the group consisting of Bacillus subtilis, Bacillus licheniformis, Herpetosiphon aurantiacus, Streptococcus pneumoniae, Chromobacterium violaceum, and Bifidobacterium adolescentis.
(10) The process of (7) above, wherein the microorganism which has an ability to produce the protein of (1) is the transformant according to any one of (4) to (6).
(11) A process for producing a peptide, which comprises allowing the protein of (1), one or more kinds of substrates selected from among an amino acid, an amino acid derivative and a peptide, and adenosine-5′-triphosphate (hereinafter, abbreviated as ATP) to be present in an aqueous medium, allowing the peptide to form and accumulate in the medium, and then recovering the peptide from the medium.
(12) A process for producing a peptide, which comprises allowing a culture or a treated culture of a microorganism which has an ability to produce the protein of (1), and one or more kinds of substrates selected from among an amino acid, an amino acid derivative, and a peptide to be present in an aqueous medium, allowing the peptide to form and accumulate in the medium, and then recovering the peptide from the medium.
(13) The process of (12) above, wherein the microorganism which has an ability to produce the protein of (1) is the transformant according to any one of (4) to (6).
(14) The process of (12) above, wherein the microorganism which has an ability to produce the protein of (1) belongs to the genus Bacillus, the genus Herpetosiphon, the genus Streptococcus, the genus Chromobacterium, or the genus Bifidobacterium.
(15) The process of (14) above, wherein the microorganism belonging to the genus Bacillus, the genus Herpetosiphon, the genus Streptococcus, the genus Chromobacterium, or the genus Bifidobacterium belongs to species selected from the group consisting of Bacillus subtilis, Bacillus licheniformis, Herpetosiphon aurantiacus, Streptococcus pneumoniae, Chromobacterium violaceum, and Bifidobacterium adolescentis.
(16) The process of (12) to (15) above, wherein the treated culture is a concentrate of a culture, a dried culture, a cell obtained by centrifugation of a culture, a dried product of the cell, a freeze-dried product of the cell, the cell treated with a surfactant, an ultrasonicated product of the cell, a mechanically ground product of the cell, the cell treated with a solvent, the cell treated with an enzyme, a protein fraction of the cell, the cell in an immobilized form, or an enzymatic preparation obtained via extraction from the cell.
(17) A process for producing a peptide, which comprises culturing a microorganism which has an ability to produce a protein having peptide-synthesizing activity and has an ability to produce at least one kind of substrate selected from among an amino acid, an amino acid derivative, and a peptide in a medium, allowing the peptide to form and accumulate in the medium, and then recovering the peptide from the medium.
(18) The process of (17) above, wherein the protein having peptide-synthesizing activity is the protein of (1).
(19) The process of (18) above, wherein the microorganism which has an ability to produce the protein of (1) and has an ability to produce at least one kind of substrate selected from among an amino acid, an amino acid derivative, and a peptide is the transformant of any one of (4) to (6).

### Effects of the Invention

According to the present invention, a protein having peptide-synthesizing activity can be produced. Furthermore, a peptide can be produced using the protein or a transformant or a microorganism which has an ability to produce the protein.

### BEST MODES FOR CARRYING OUT THE INVENTION

### 1. Protein of the Present Invention

Examples of the protein of the present invention include:
[1] a protein having the amino acid sequence shown by any one selected from the group consisting of SEQ ID NOS: 2, 4, 6, 8, 10, and 12;
[2] a protein consisting of the amino acid sequence that has a deletion, a substitution, or an addition of one or more amino acids in the amino acid sequence shown by any one selected from the group consisting of SEQ ID NOS: 2, 4, 6, 8, 10, and 12, and having peptide-synthesizing activity; and
[3] a protein consisting of an amino acid sequence that has 80% or more homology with the amino acid sequence shown by any one selected from the group consisting of SEQ ID NOS: 2, 4, 6, 8, 10, and 12, and having peptide-synthesizing activity.

The above protein consisting of an amino acid sequence that has a deletion, a substitution, or an addition of one or more amino acids and having peptide-synthesizing activity can be obtained by site-directed mutagenesis according to Molecular Cloning, A Laboratory Manual, 2nd Edition Cold Spring Harbor Laboratory Press (1989) (hereinafter, abbreviated as Molecular Cloning, 2nd Edition), Current Protocols in Molecular Biology, John Wiley & Sons (1987-1997) (hereinafter, abbreviated as Current Protocols In Molecular Biology), Nucleic Acids Research, 10, 6487 (1982), Proc. Natl. Acad. Sci. U.S.A., 79, 6409 (1982), Gene, 34, 315 (1985), Nucleic Acids Research, 13, 4431 (1985), Proc. Natl. Acad. Sci. U.S.A., 82, 488 (1985), or the like. For example, the protein can be obtained by introducing site-specific mutation into a DNA encoding a protein consisting of the amino acid sequence shown by SEQ ID NO: 2.

The number of amino acids to be deleted, substituted, or added is not particularly limited, but is the number such that deletion, substitution, or addition can be carried out by a known method such as the above site-specific mutagenesis. The number of amino acids ranges from 1 to dozens, preferably 1 to 20, more preferably 1 to 10, and further more preferably 1 to 5.

The phrase “a deletion, a substitution, or an addition of one or more amino acids in the amino acid sequence shown by SEQ ID NO: 2, 4, 6, 8, 10, or 12” means that, at any position in such sequence, 1 or a plurality of amino acids may be deleted, substituted, or added.

Moreover, examples of amino acid positions in which amino acid deletion, substitution, or addition can be introduced include one to several amino acid positions at the N-terminus and the C-terminus of the amino acid sequence shown by SEQ ID NO: 2, 4, 6, 8, 10, or 12.

A deletion, a substitution, and an addition may be introduced simultaneously. Amino acids to be substituted or added may be natural or unnatural. Examples of natural amino acids include L-alanine, L-asparagine, L-aspartic acid, L-arginine, L-glutamine, L-glutamic acid, glycine, L-histidine, L-isoleucine, L-leucine, L-lysine, L-methionine, L-phenylalanine, L-proline, L-serine, L-threonine, L-tryptophan, L-tyrosine, L-valine, and L-cysteine.

Examples of amino acids that are mutually substitutable are listed below. Amino acids in the same group can be substituted with each other.

Group A: leucine, isoleucine, norleucine, valine, norvaline, alanine, 2-aminobutanoic acid, methionine, O-methylserine, t-butylglycine, t-butylalanine, and cyclohexylalanine

Group B: aspartic acid, glutamic acid, isoaspartic acid, isoglutamic acid, 2-aminoadipic acid, and 2-aminosuberic acid

Group C: asparagine and glutamine

Group D: lysine, arginine, ornithine, 2,4-diaminobutanoic acid, and 2,3-diaminopropionic acid

Group E: proline, 3-hydroxyproline, and 4-hydroxyproline

Group F: serine, threonine, and homoserine

Group G: phenylalanine and tyrosine

Furthermore, the protein of the present invention desirably has 80% or more, preferably 90% or more, more preferably 95% or more, further more preferably 98% or more, and particularly preferably 99% or more homology with the amino acid sequence shown by any one of SEQ ID NOS: 2, 4, 6, 8, 10, and 12, so that the protein has peptide-synthesizing activity.

Amino acid sequence homology or nucleotide sequence homology can be determined using the algorithm BLAST of Karlin and Altschul [Pro. Natl. Acad. Sci. U.S.A., 90, 5873(1993)] or FASTA [Methods Enzymol., 183, 63 (1990)]. Based on the algorithm BLAST, a program called BLASTN or BLASTX has been developed [J. Mol. Biol., 215, 403(1990)]. When nucleotide sequences are analyzed by BLASTN based on BLAST, parameters such as Score=100 and word length=12 are employed. Also, when amino acid sequences are analyzed by BLASTX based on BLAST, parameters such as score=50 and word length=3 are employed. When BLAST and Gapped BLAST program are employed, default parameters of each program are used. Specific techniques for these analytical methods are known (http://www.ncbi.nlm.nih.gov.).

Furthermore, a protein consisting of an amino acid sequence having 80% or more, preferably 90% or more, more preferably 95% or more, further more preferably 98% or more, and particularly preferably 99% or more homology with the amino acid sequence shown by SEQ ID NO: 2, 4, 6, 8, 10, or 12, and having peptide-synthesizing activity is also the protein of the present invention. Amino acid sequence homology can be determined using BLAST and FASTA as described above.

The term “peptide-synthesizing activity” in the present invention refers to activity of forming a peptide having a length that is the same as or longer than that of a dipeptide using an amino acid, an amino acid derivative, a peptide comprising them, or the like as a substrate and adenosine-5′-triphosphate (hereinafter, abbreviated as ATP). Preferably the “peptide-synthesizing activity” refers to activity of forming a peptide having a length that is the same as or longer than that of a dipeptide as a result of repetition (one or more times) of a reaction for binding an L-amino acid, glycine, or an L-amino acid derivative having no modification group at a carboxyl group to an L-amino acid, glycine, or an L-amino acid derivative, the N-terminus of a peptide comprising L-amino acids, glycines, or L-amino acid derivatives.

An example of a method for confirming that the protein of the present invention is a protein having peptide-synthesizing activity involves preparing a transformant that expresses the protein of the present invention by a DNA recombination method, producing the protein of the present invention using the transformant, allowing the thus purified protein as an enzyme source, one or more kinds of substrate selected from among amino acids, amino acid derivatives and peptides, and ATP to be present in an aqueous medium, and then analyzing by HPLC or the like whether or not a peptide is formed and accumulated in the aqueous medium.

### 2. DNA of the Present Invention

Examples of the DNA of the present invention include:
[1] a DNA encoding the protein according to claim 1;
[2] a DNA having a nucleotide sequence shown by any one selected from the group consisting of SEQ ID NOS: 1, 3, 5, 7, 9, and 11; and
[3] a DNA hybridizing under stringent conditions to a DNA consisting of a nucleotide sequence complementary to the nucleotide sequence shown by any one selected from the group consisting of SEQ ID NOS: 1, 3, 5, 7, 9, and 11, and encoding a protein having peptide-synthesizing activity.

Here the term “hybridizing” refers to a step such that a DNA hybridizes to a DNA having a specific nucleotide sequence or a part of the DNA. Therefore, the DNA having a specific nucleotide sequence or a nucleotide sequence of a part of the DNA is useful as a probe for Northern or Southern blot analysis or may be a DNA with a length such that the DNA can be used as an oligonucleotide primer for PCR analysis. An example of a DNA to be used as a probe for Northern or Southern blot analysis is a DNA with a length of at least 100 nucleotides or more, preferably 200 nucleotides or more, and more preferably 500 nucleotides or more. An example of a DNA to be used as an oligonucleotide primer is a DNA with a length of at least 10 nucleotides or more and preferably 15 nucleotides or more.

Experimental methods for DNA hybridization are known well. For example, hybridization conditions are determined according to Molecular Cloning 2nd Edition, 3rd Edition (2001), Methods for General and Molecular Bacteriology, ASM Press (1994), and Immunology methods manual, Academic press (Molecular) in addition to many other standard textbooks and then an experiment can be conducted.

The above stringent conditions are, for example, conditions under which a DNA-immobilized filter and a probe DNA are incubated overnight at 42° C. in a solution containing 50% formamide, 5×SSC (750 mM sodium chloride, 75 mM/L sodium citrate), 50 mM sodium phosphate (pH 7.6), 5× Denhardt's solution, 10% dextran sulfate, and denatured 20 μg/L salmon sperm DNA, following which the filter is washed in a 0.2×SSC solution at approximately 65° C., for example. Less stringent conditions can also be employed herein. Stringent conditions can be varied via adjustment of formamide concentration (the lower the formamide concentration, the less the stringency), change of salt concentration, and change of temperature conditions. Such less stringent conditions are, for example, conditions under which incubation is carried out overnight at 37° C. in a solution containing 6×SSCE (20×SSCE: 3 mol/L sodium chloride, 0.2 mol/L sodium dihydrogenphosphate, 0.02 mol/L EDTA, and pH 7.4), 0.5% SDS, 30% formamide, and denatured 100 μg/L salmon sperm DNA, followed by washing using 1×SSC and 0.1% SDS solutions at 50° C. Even less stringent conditions are, for example, the above less stringent conditions under which hybridization is carried out using a solution with a high salt concentration (e.g., 5×SSC), followed by washing.

Various conditions described above can also be set by adding or changing a blocking reagent to be used to suppress the background conditions of a hybridization experiment. Such addition of a blocking reagent may be associated with changes in hybridization conditions, and may thus be used to adjust the conditions.

An example of a DNA that can hybridize under the above-mentioned stringent conditions is a DNA having at least 80% or more, preferably 90% or more, more preferably 95% or more, further more preferably 98% or more, and particularly preferably 99% or more homology with the nucleotide sequence of the DNA of any one of [1] to [3] above, when calculation is performed based on the above parameters using the above program such as BLAST or FASTA.

Nucleotide sequence homology can be determined using the above-mentioned program such as BLAST or FASTA.

That a DNA hybridizing to the above DNA under stringent conditions is a DNA encoding a protein having peptide-synthesizing activity can be confirmed by a method that involves preparing a recombinant DNA that expresses the DNA, purifying the relevant protein from a culture that is obtained by culturing a microorganism (the microorganism is obtained by introducing the recombinant DNA into host cells), and then allowing the purified protein (using as an enzyme source), and one or more kinds of amino acids, amino acid derivatives, or peptides to be present in an aqueous medium, and then analyzing by HPLC or the like whether or not a peptide is formed and accumulated in the aqueous medium.

### 3. Microorganisms and Transformants to be Used for the Process of the Present Invention

Microorganisms and transformants to be used for the process of the present invention are not particularly limited as long as they are microorganisms and transformants which have an ability to produce the protein of the present invention. Examples of such microorganisms include preferably microorganisms belonging to the genus Bacillus, the genus Herpetosiphon, the genus Streptococcus, the genus Chromobacterium, or the genus Bifidobacterium, preferably microorganisms belonging to Bacillus subtilis, Bacillus licheniformis, Herpetosiphon aurantiacus, Streptococcus pneumoniae, Chromobacterium violaceum, or Bifidobacterium adolescentis, and more preferably Bacillus subtilis ATCC6633 and Bacillus licheniformis ATCC14580, Streptococcus pneumoniae ATCC BAA-255, Herpetosiphon aurantiacus ATCC 23779, Chromobacterium violaceum NBRC 12614, and Bifidobacterium adolescentis JCM1275. Examples of such transformants include transformants obtained via transformation with a DNA encoding the protein of the present invention.

Examples of transformants obtained via transformation with a DNA encoding the protein of the present invention include transformants obtained via transformation of host cells with a recombinant DNA containing the DNA in 2 above according to a known method. Examples of host cells include prokaryotes such as bacteria, yeast, animal cells, insect cells, and plant cells, preferably prokaryotes such as bacteria or yeast, more preferably prokaryotes such as bacteria, and more preferably microorganisms belonging to the genus Escherichia.

### 4. Preparation of the DNA of the Present Invention

The DNA of the present invention can be obtained by: carrying out Southern hybridization for a chromosomal DNA library of a microorganism, which has the DNA of the present invention on the chromosome, such as a microorganism belonging to the genus Bacillus, the genus Herpetosiphon, the genus Streptococcus, the genus Chromobacterium, or the genus Bifidobacterium, with the use of probes and primers that can be designed based on the nucleotide sequence shown by SEQ ID NO: 1, 3, 5, 7, 9, or 11. Alternatively, the DNA can be obtained by carrying out a technique such as PCR using the chromosome of a microorganism (descrived above) having the DNA of the present invention on the chromosome.

The DNA of the present invention can be obtained by carrying out Southern hybridization for a chromosomal DNA library of a microorganism belonging to the genus Bacillus, preferably a microorganism belonging to Bacillus subtilis, and more preferably Bacillus subtilis ATCC6633 using a probe that can be designed based on the nucleotide sequence shown by SEQ ID NO: 1, for example. Alternatively, the DNA can be obtained by carrying out PCR [PCR Protocols, Academic Press (1990)] using primer DNAs that can be designed based on the nucleotide sequence shown by SEQ ID NO: 1 and a chromosomal DNA of a microorganism, preferably a microorganism belonging to the genus Bacillus, more preferably a microorganism belonging to Bacillus subtilis, and further more preferably Bacillus subtilis ATCC6633 as a template.

Moreover, various gene sequence databases are searched for a sequence having 85% or more, preferably 90% or more, more preferably 95% or more, further more preferably 98% or more, and particularly preferably 99% or more homology with the nucleotide sequence of a DNA encoding the amino acid sequence shown by SEQ ID NO: 1, 3, 5, 7, 9, or 11. Based on the nucleotide sequence obtained by the search, the DNA of the present invention or a DNA to be used in the process of the present invention can also be obtained by the above-mentioned method from chromosomal DNAs, cDNA libraries, and the like of organisms having the nucleotide sequence.

The nucleotide sequence of the thus obtained DNA can be determined by directly incorporating the DNA into or digesting the DNA with an appropriate restriction enzyme or the like and then incorporating into a vector by a conventional method, introducing the thus obtained recombinant DNA into host cells, and then analyzing the resultant using a generally employed nucleotide sequence analysis method, such as a dideoxy chain termination method [Proc. Natl. Acad. Sci., U.S.A., 74, 5463 (1977)] or a nucleotide sequence analyzer such as an ABI3700 DNA analyzer (Applied Biosystems).

When the thus obtained DNA is a partial-length DNA as revealed by determination of the nucleotide sequence, the full-length DNA can be obtained by carrying out Southern hybridization or the like for a chromosomal DNA library using the partial-length DNA as a probe.

Moreover, based on the thus determined nucleotide sequence of the DNA, a target DNA can also be prepared by chemical synthesis using a Model 8905 DNA Synthesizer (Perseptive Biosystems) or the like.

An example of a DNA obtained as described above is a DNA having the nucleotide sequence shown by SEQ ID NO: 1.

Examples of a vector into which the DNA of the present invention is incorporated include pBluescriptII KS(+) (Stratagene), pDIRECT [Nucleic Acids Res., 18, 6069 (1990)], pCR-Script Amp SK(+) (Stratagene), pT7Blue (Novagen), pCR II (Invitrogen Corporation), and pCR-TRAP (GeneHunter).

Examples of host cells include microorganisms belonging to the genus Escherichia. Examples of such microorganisms belonging to the genus Escherichia include Escherichia coli XL1-Blue, Escherichia coli XL2-Blue, Escherichia coli DH1, Escherichia coli MC1000, Escherichia coli ATCC 12435, Escherichia coli W1485, Escherichia coli JM109, Escherichia coli HB101, Escherichia coli No.49, Escherichia coli W3110, Escherichia coli NY49, Escherichia coli MP347, Escherichia coli NM522, Escherichia coli BL21, and Escherichia coli ME8415.

Any method can be used as a method for introducing a recombinant DNA, as long as it is a method for introducing a DNA into the above host cells. Examples of such method include a method using calcium ions [Proc. Natl. Acad. Sci., U.S.A., 69, 2110 (1972)], a protoplast method (JP Patent Publication (Kokai) No. 63-248394 (1988)), and electroporation [Nucleic Acids Res., 16, 6127 (1988)].

An example of the transformant of the present invention obtained by the above method is Escherichia coli BL21 (DE3)/pRBS that is a microorganism containing a recombinant DNA containing a DNA having the nucleotide sequence shown by SEQ ID NO: 1.

### 5. Process for Producing Transformants and Microorganisms to be Used for the Pocesses of the Present Invention

Based on the DNA of the present invention, a DNA fragment with an appropriate length containing a part that encodes the protein of the present invention is prepared, as necessary. Also, the nucleotide sequence of a part that encodes the protein is subjected to nucleotide substitution so as to prepare an optimum codon for expression in a host. As a result, a transformant with an improved productivity of the protein can be obtained.

A recombinant DNA is prepared by inserting the DNA fragment downstream of a promoter of an appropriate expression vector.

The thus prepared recombinant DNA is introduced into host cells appropriate for the expression vector, so that a transformant that produces the protein of the present invention can be obtained.

Any host cells can be used, as long as they can express a target gene, such as prokaryotes (e.g., bacteria), yeast, animal cells, insect cells, and plant cells.

Expression vectors to be used herein can autonomously replicate within the host cells or can be incorporated into a chromosome and contains a promoter at a position into which the DNA of the present invention can be transcribed.

When a prokaryote such as a bacterium is used as a host cell, a recombinant DNA having the DNA of the present invention is preferably autonomously replicable in a prokaryote, and containing a promoter, a ribosome binding sequence, the DNA of the present invention, and a transcription termination sequence. A gene controlling a promoter may also be contained.

Examples of expression vectors include pBTrp2, pBTac1, pBTac2, pHelix1 (all of them are manufactured by Roche Diagnostics K.K.), pKK233-2 (Amersham•Pharmacia Biotech), pSE280 (Invitrogen Corporation), pGEMEX-1 (Promega), pQE-8 (QIAGEN), pET-3 (Novagen), pKYP10 (JP Patent Publication (Kokai) No. 58-110600 A (1983)), pKYP200 [Agric. Biol. Chem., 48, 669 (1984)], pLSA1 [Agric. Biol. Chem., 53, 277 (1989)], pGEL1 [Proc. Natl. Acad. Sci., U.S.A., 82, 4306 (1985)], pBluescriptII SK(+), pBluescript II KS(−) (Stratagene), pTrS30 [prepared from Escherichia coli JM109/pTrS30 (FERM BP-5407)], pTrS32 [prepared from Escherichia coli JM109/pTrS32 (FERM BP-5408)], pPAC31 (WO98/12343), pUC19 [Gene, 33, 103 (1985)], pSTV28 (Takara Bio Inc.), pUC118 (Takara Bio Inc.), and pPA1 (JP Patent Publication (Kokai) No. 63-233798 A (1988)).

As a promoter, any promoter may be used herein, as long as it functions within host cells such as Escherichia coli. Examples of such promoter include promoters derived from Escherichia coli or a phage, such as a trp promoter (Pₜᵣₚ), a lac promoter (P_{lac}), a P_{L} promoter, a P_{R} promoter, and a P_{SE} promoter, an SPO1 promoter, an SPO2 promoter, and a penP promoter. Furthermore, artificially designed and altered promoters and the like can also be used herein, such as promoters in which two Pₜᵣₚ are arranged in tandem, a tac promoter, a lacT7 promoter, and a let I promoter.

Moreover, an xylA promoter [Appl. Microbiol. Biotechnol., 35, 594-599 (1991)] to be expressed in a microorganism belonging to the genus Bacillus, a P54-6 promoter [Appl. Microbiol. Biotechnol., 53, 674-679 (2000)] to be expressed in a microorganism belonging to the genus Corynebacterium, or the like can also be used.

Furthermore, a plasmid, in which the distance between Shine-Dalgarno sequence that is a ribosome binding sequence and the initiation codon is appropriately regulated (e.g., 6 to 18 nucleotides) is preferably used.

In a recombinant DNA in which the DNA of the present invention is ligated to an expression vector, a transcription termination sequence is not always required, but a transcription termination sequence is preferably arranged immediately following a structural gene.

An example of such recombinant DNA is pBsRz.

Examples of prokaryotes include microorganisms belonging to the genus Escherichia, the genus Bacillus, the genus Brevibacterium, the genus Corynebacterium, the genus Microbacterium, the genus Serratia, the genus Pseudomonas, the genus Agrobacterium, the genus Alicyclobacillus, the genus Anabena, the genus Anacystis, the genus Arthrobacter, the genus Azotobacter, the genus Chromatium, the genus Erwinia, the genus Methylobacterium, the genus Phormidium, the genus Rhodobacter, the genus Rhodopseudomonas, the genus Rhodospirillum, the genus Scenedesmus, the genus Streptomyces, the genus Synechoccus, and the genus Zymomonas. Specific examples of such microorganisms include Escherichia coli XL1-Blue, Escherichia coli XL2-Blue, Escherichia coli DH1, Escherichia coli DH5α, Escherichia coli MC1000, Escherichia coli KY3276, Escherichia coli W1485, Escherichia coli JM109, Escherichia coli HB101, Escherichia coli No. 49, Escherichia coli W3110, Escherichia coli NY49, Escherichia coli MP347, Escherichia coli NM522, Escherichia coli BL21, Bacillus subtilis ATCC33712, Bacillus megaterium, Brevibacterium ammoniagenes, Brevibacterium immariophilum ATCC14068, Brevibacterium saccharolyticum ATCC 14066, Brevibacterium flavum ATCC 14067, Brevibacterium lactofermentum ATCC13869, Corynebacterium glutamicum ATCC13032, Corynebacterium glutamicum ATCC14297, Corynebacterium acetoacidophilum ATCC13870, Microbacterium ammoniaphilum ATCC15354, Serratia ficaria, Serratia fonticoliz, Serratia liquefaciens, Serratia marcescens, Pseudomonas sp. D-0110, Agrobacterium radiobacter, Agrobacterium rhizogenes, Agrobacterium rubi, Anabaena cylindrica, Anabaena doliolum, Anabaena flos-aquae, Arthrobacter aurescens, Arthrobacter citreus, Arthrobacter globformis, Arthrobacter hydrocarboglutamicus, Arthrobacter mysorens, Arthrobacter nicotianae, Arthrobacter paraffineus, Arthrobacter protophormiae, Arthrobacter roseoparaffinus, Arthrobacter sulfureus, Arthrobacter ureafaciens, Chromatium buderi, Chromatium tepidum, Chromatium vinosum, Chromatium warmingii, Chromatium fluviatile, Erwinia uredovora, Erwinia carotovora, Erwinia ananas, Erwinia herbicola, Erwinia punctata, Erwinia terreus, Methylobacterium rhodesianum, Methylobacterium extorquens, Phormidium sp. ATCC29409, Rhodobacter capsulatus, Rhodobacter sphaeroides, Rhodopseudomonas blastica, Rhodopseudomonas marina, Rhodopseudomonas palustris, Rhodospirillum rubrum, Rhodospirillum salexigens, Rhodospirillum salinarum, Streptomyces ambofaciens, Streptomyces aureofaciens, Streptomyces aureus, Streptomyces fungicidicus, Streptomyces griseochromogenes, Streptomyces griseus, Streptomyces lividans, Streptomyces olivogriseus, Streptomyces rameus, Streptomyces tanashiensis, Streptomyces vinaceus, and Zymomonas mobilis.

Any method can be used as a method for introducing a recombinant DNA, as long as it is a method for introducing a DNA into the above host cells. Examples of such method include a method using calcium ions [Proc. Natl. Acad. Sci., U.S.A., 69, 2110 (1972)], a protoplast method (JP Patent Publication (Kokai) No. 63-248394 A (1988)), and electroporation [Nucleic Acids Res., 16, 6127 (1988)].

When yeast is used as a host cell, YEp13 (ATCC37115), YEp24 (ATCC37051), YCp50 (ATCC37419), pHS19, pHS15, or the like can be used as an expression vector.

Any promoter can be used as long as it functions within a yeast strain. Examples of such promoter include a PHO5 promoter, a PGK promoter, a GAP promoter, an ADH promoter, a gal 1 promoter, a gal 10 promoter, a heat shock polypeptide promoter, an MFα1 promoter, and a CUP 1 promoter.

Examples of yeast include yeast strains belonging to the genus Saccharomyces, the genus Schizosaccharomyces, the genus Kluyveromyces, the genus Trichosporon, the genus Schwanniomyces, the genus Pichia, and the genus Candida. Specific examples of such yeast strains include Saccharomyces cerevisiae, Schizosaccharomyces pombe, Kluyveromyces lactis, Trichosporon pullulans, Schwanniomyces alluvius, Pichia pastoris, and Candida utilis.

Any method for introducing a recombinant DNA can be used, as long as it is a method for introducing a DNA into yeast. Examples of such method include electroporation [Methods Enzymol., 194, 182 (1990)], the spheroplast method [Proc. Natl. Acad. Sci., U.S.A., 81, 4889 (1984)], and the lithium acetate method [J. Bacteriol., 153, 163 (1983)].

When animal cells are used as hosts, examples of expression vectors that can be used herein include pcDNAI, pcDM8 (commercially available from Funakoshi Corporation), pAGE107 (JP Patent Publication (Kokai) No. 3-22979 A (1991)), pAS3-3 (JP Patent Publication (Kokai) No. 2-227075 A (1990)), pCDM8 [Nature, 329, 840 (1987)], pcDNAI/Amp (Invitrogen Corporation), pREP4 (Invitrogen Corporation), pAGE103 [J. Biochem, 101, 1307 (1987)], pAGE210, pAMo, and pAMoA.

Any promoter can be used, as long as it functions within animal cells. Examples of such promoter include a cytomegalovirus (CMV) IE (immediate early) gene promoter or SV40 early promoter, a metallothionein promoter, a retroviral promoter, a heat shock promoter, and an SRα promoter. Moreover, a human CMV IE gene enhancer can be used together with a promoter.

Examples of animal cells include mouse myeloma cells, rat myeloma cells, mouse hybridoma cells, Namalwa cells or Namalwa KJM-1 cells that are human cells, human fetal kidney cells, human leukemia cells, African green monkey kidney cells, CHO cells that are Chinese hamster cells, and HBT5637 (JP Patent Publication (Kokai) No. 63-299 A (1988)).

Examples of mouse myeloma cells include SP2/0 and NSO. Examples of rat myeloma cells include YB2/0. Examples of human fetal kidney cells include HEK293 (ATCC CRL-1573). Examples of human leukemia cells include BALL-1. Examples of African green monkey kidney cells include COS-1 and COS-7.

Any method can be used for introducing a recombinant DNA, as long as it is a method for introducing DNA into animal cells. Examples of such method include electroporation [Cytotechnology, 3, 133 (1990)], a calcium phosphate method (JP Patent Publication (Kokai) No. 2-227075 A (1990)), lipofection [Proc. Natl. Acad. Sci., U.S.A., 84, 7413 (1987)], and a method described in Virology, 52, 456 (1973).

When insect cells are used as hosts, a protein can be produced by a method described in Baculovirus Expression Vectors, A Laboratory Manual, W. H. Freeman and Company, New York (1992), Current Protocols in Molecular Biology, Molecular Biology, A Laboratory Manual, Bio/Technology, 6, 47 (1988), or the like.

Specifically, a recombinant gene transfer vector and baculovirus are co-introduced into insect cells and then a recombinant virus is obtained in a supernatant obtained by culturing insect cells. The insect cells are further infected with the recombinant virus, so that the protein can be produced.

Examples of such gene transfer vector to be used in the method include pVL1392, pVL1393, and pBlueBacIII (all of them are manufactured by Invitrogen Corporation).

As baculovirus, Autographa californica nuclear polyhedrosis virus that infects insects of the genus Mamestra (the subfamily Hadeninae, the family Noctuidae) can be used, for example.

As insect cells, ovary cells of Spodoptera frugiperda, ovary cells of Trichoplusia ni, cultured cells derived from silkworm ovary, and the like can be used.

Examples of ovary cells of Spodoptera frugiperda include Sf9 and Sf21 (Baculovirus Expression Vectors: A Laboratory Manual). Examples of ovary cells of Trichoplusia ni include High 5 and BTI-TN-5B1-4 (Invitrogen Corporation). Examples of cultured cells derived from silkworm ovary include Bombyx mori N4 and the like.

Examples of a method for co-introduction of the above recombinant gene transfer vector and the above baculovirus into insect cells for preparation of a recombinant virus include a calcium phosphate method (JP Patent Publication (Kokai) No. 2-227075 A (1990)), and lipofection [Proc. Natl. Acad. Sci., U.S.A., 84, 7413 (1987)].

When plant cells are used as host cells, examples of expression vectors include a Ti plasmid and a tobacco mosaic virus vector.

Any promoter can be used, as long as it functions within plant cells. Examples of such promoter include a cauliflower mosaic virus (CaMV) 35S promoter and a rice actin 1 promoter.

Examples of host cells include plant cells of tobacco, potato, tomato, carrot, soybean, rapeseed, alfalfa, rice, wheat, barley, and the like.

Any method for introducing a recombinant vector can be used, as long as it is a method for introducing a DNA into plant cells. Examples of such method include a method using Agrobacterium (JP Patent Publication (Kokai) No. 59-140885 A (1984), JP Patent Publication (Kokai) No. 60-70080 A (1985), WO94/00977), electroporation (JP Patent Publication (Kokai) No. 60-251887 A (1985)), a method using a particle gun (gene gun) (JP Patent No. 2606856 and JP Patent No. 2517813).

### 6. Process for Producing the Protein of the Present Invention

A transformant obtained by the method in 5 above is cultured in a medium, the protein of the present invention is formed and accumulated in the culture, and then the protein is recovered from the culture. Thus, the protein can be produced.

A host for the above transformant for production of the protein of the present invention may be any of a prokaryote, yeast, an animal cell, an insect cell, a plant cell, and the like. Specific examples of such host include preferably prokaryotes such as bacteria, more preferably microorganisms belonging to the genus Escherichia, and further more preferably microorganisms belonging to Escherichia coli.

When the protein is expressed by yeast, animal cells, insect cells, or plant cells, the protein to which a sugar or a sugar chain has been added can be obtained.

The above transformant can be cultured in a medium according to a method generally employed for culturing a host.

Both a natural medium and a synthetic medium may be used as media for culturing a transformant obtained using a prokaryote such as Escherichia coli or an eukaryote such as yeast as a host, as long as it contains a carbon source, a nitrogen source, inorganic salts, and the like that can be assimilated by the relevant organisms and enables efficient culturing of the transformant.

Any carbon source can be used herein, as long as it can be assimilated by said organisms. Examples of such carbon source include carbohydrates such as glucose, fructose, sucrose, molasses containing them, and starch or starch hydrolysate, organic acids such as acetic acid and propionic acid, and alcohols such as ethanol and propanol.

Examples of a nitrogen source that can be used herein include ammonia, ammonium salts of inorganic acids or organic acids such as ammonium chloride, ammonium sulfate, ammonium acetate, and ammonium phosphate, other nitrogen-containing compounds, and, peptone, a meat extract, a yeast extract, corn steep liquor, casein hydrolysate, soybean cake and soybean cake hydrolysate, various fermentation microbes, and digests thereof.

As inorganic salts, monopotassium phosphate, dipotassium phosphate, magnesium phosphate, magnesium sulfate, sodium chloride, ferrous sulfate, manganese sulfate, copper sulfate, calcium carbonate, and the like can be used.

Culturing is generally carried out under aerobic conditions by shaking culture, deep aeration stirring culture, or the like. The culturing temperature preferably ranges from 15° C. to 40° C. The culturing time generally ranges from 5 hours to 7 days. The pH during culturing is maintained between 3.0 and 11. The pH is adjusted using inorganic or organic acid, an alkaline solution, urea, calcium carbonate, ammonia, or the like.

Also, an antibiotic such as ampicillin or tetracycline may be added to a medium during culturing if necessary.

When a microorganism transformed with an expression vector containing an inducible promoter is cultured, an inducer may be added to a medium as necessary. For example, when a microorganism transformed with an expression vector containing a lac promoter is cultured, isopropyl-β-D-thiogalactopyranoside or the like may be added to the medium. When a microorganism transformed with an expression vector containing a trp promoter is cultured, indoleacrylic acid or the like may be added to the medium.

As a medium for culturing a transformant obtained using an animal cell as a host, a generally employed RPMI1640 medium [J. Am. Med. Assoc., 199, 519 (1967)], Eagle's MEM medium [Science, 122, 501 (1952)], DMEM medium [Virology, 8, 396 (1959)], or 199 medium [Proc. Soc. Biol. Med., 73, 1 (1950)] or such medium supplemented with fetal calf serum or the like can be used.

Culturing is generally carried out under conditions of pH6 to 8, 25° C. to 40° C., the presence of 5% CO₂, and the like for 1 to 7 days.

Also, an antibiotic such as kanamycin, penicillin, or streptomycin may be added to a medium during culturing, as necessary.

As a medium for culturing a transformant obtained using an insect cell as a host, a generally employed TNM-FH medium (Pharmingen), Sf-900 II SFM medium (Life Technologies), ExCell400, ExCell405 [all of them are manufactured by JRH Biosciences], Grace's Insect Medium [Nature, 195, 788 (1962)], or the like can be used.

Culturing is generally carried out under conditions of pH6 to 7, 25° C. to 30° C., and the like for 1 to 5 days.

Also, an antibiotic such as gentamicin may be added to a medium during culturing, as necessary.

A transformant obtained using a plant cell as a host can be cultured as cells or caused to differentiate into plant cells or organs and then cultured. As a medium for culturing the transformant, a generally employed Murashige and Skoog (MS) medium, White medium, or such medium supplemented with a plant hormone such as auxin or cytokinin can be used.

Culturing is generally carried out under conditions of pH5 to 9, 20° C. to 40° C., and for 3 to 60 days.

Also, an antibiotic such as kanamycin or hygromycin may be added to a medium during culturing, as necessary.

Examples of the process for producing the protein of the present invention include a process that comprises allowing the protein produced within host cells, a process that comprises allowing the protein to be secreted outside the host cells, and a process that comprises allowing the protein to be produced on the extracellular membranes of the host cells. The structure of the protein to be produced can be varied depending on the selected method.

When the protein of the present invention is produced within host cells or on the extracellular membranes of host cells, the protein can be actively secreted by the host cells extracellularly via application of the method of Paulson et al [J. Biol. Chem., 264, 17619 (1989)], the method of Row et al [Proc. Natl. Acad. Sci., U.S.A., 86, 8227 (1989), Genes Develop., 4, 1288(1990)], or the method described in JP Patent Publication (Kokai) No. 05-336963 A (1993), WO94/23021, or the like.

Specifically, with the use of a gene recombination technique, a protein containing the active site of the protein of the present invention is produced in a form such that a signal peptide is added in front of the protein. Hence, the protein can be actively secreted outside the host cells.

Moreover, according to the method described in JP Patent Publication (Kokai) No. 2-227075 A (1990), productivity can also be increased using a gene amplification system using a dihydrofolate reductase gene or the like.

Furthermore, gene-transferred animal or plant cells are caused to redifferentiate, so that animal individuals (transgenic non-human animals) or plant individuals (transgenic plants) into which a gene has been introduced are produced and the protein of the present invention can also be produced using these animal individuals or plant individuals.

When a transformant producing the protein of the present invention is an animal or a plant individual, such transformant is raised or cultivated according to a general method and then allowed to form and accumulate the protein. The protein is recovered from the animal individual or the plant individual, so that the protein can be produced.

An example of the process for producing the protein of the present invention using an animal individual is a process by which the protein of the present invention is produced in animals that have been produced by introducing a gene according to a known method [Am. J. Clin. Nutr., 63, 639S (1996), Am. J. Clin. Nutr., 63, 627S (1996), Bio/Technology, 9, 830 (1991)].

In the case of animal individuals, the protein of the present invention can be produced by raising transgenic non-human animals in which the DNA of the present invention or a DNA to be used in the process of the present invention has been introduced, allowing the protein to form and accumulate in the animals, and then recovering the protein from the animals, for example. Examples of places in which the protein is formed and accumulated in animals include the milk of animals (JP Patent Publication (Kokai) No. 63-309192 A (1988)) and eggs. Any promoter can be used in this case, as long as it functions within animals. Examples of such promoter that is preferably used include mammary-cell-specific promoters such as an α casein promoter, a β casein promoter, a β lactoglobulin promoter, and a whey acid protein promoter.

An example of a process for producing the protein of the present invention using a plant individual is a process that involves cultivating a transgenic plant (into which a DNA encoding the protein of the present invention has been introduced) according to a known method [Tissue Culture, 20 (1994), Tissue Culture, 21 (1995), Trends Biotechnol., 15, 45 (1997)], allowing the protein to form and accumulate in the plant, and then recovering the protein from the plant, so as to produce the protein.

As a method for isolating and purifying the protein of the present invention produced using a transformant that produces the protein of the present invention, a general isolation and purification method for enzymes can be employed.

For example, when the protein of the present invention is produced in a soluble form within the cells, after completion of culturing, the cells are collected by centrifugation, suspended in an aqueous buffer, and then disrupted using a sonicator, French press, Manton-Gaulin homogenizer, dyno mill, or the like, thereby obtaining a cell free extract.

A purified proteins can be obtained from supernatants obtained by centrifugation of the cell free extracts using one of or a combination of general enzyme isolation and purification techniques, that is, solvent extraction, a salting-out method using ammonium sulfate or the like, demineralization, precipitation using an organic solvent, anion exchange chromatography using a resin such as diethylaminoethyl (DEAE)-Sepharose or DIAION HPA-75 (Mitsubishi Chemical Corporation), cation exchange chromatography using a resin such as S-Sepharose FF (Pharmacia), hydrophobic chromatography using a resin such as butyl Sepharose or phenyl Sepharose, gel filtration using a molecular sieve, affinity chromatography, electrophoresis such as chromatofocusing and isoelectric focusing, and the like.

Furthermore, when the protein is produced in an insoluble form produced within cells, cells are collected and disrupted similarly, and then centrifuged to obtain a precipitated fraction. The protein is then recovered from the fraction by a general method and then the protein of an insoluble form is solubilized using a protein denaturation agent.

The solution obtained by solubilization is diluted or dialyzed to prepare a dilute solution such that it contains no protein denaturation agent or the concentration of the protein denaturation agent is low enough so that the protein is not denatured. Thus, the protein is formed to have a normal conformation, and then a purified protein can be obtained by isolation and purification techniques similar to the above.

When the protein of the present invention or a derivative such as a sugar-modified product thereof is secreted extracellularly, the protein or a derivative such as a glycosylated product thereof can be recovered in the supernatant of the culture.

Specifically, the culture is treated by a technique similar to the above such as centrifugation, so as to obtain a soluble fraction. A purified protein can be obtained from the soluble fraction using isolation and purification techniques similar to the above.

An example of the thus obtained protein is a protein having the amino acid sequence shown by SEQ ID NOS: 2, 4, 6, 8, 10, or 12.

Also, the protein of the present invention is produced in the form of a fusion protein with another protein and then the protein can also be purified by affinity chromatography using a substance that has affinity for the protein of interest fused to the other protein. For example, according to the method of Row et al [Proc. Natl. Acad. Sci., U.S.A., 86, 8227 (1989), Genes Develop., 4, 1288 (1990)] or the method described in JP Patent Publication (Kokai) No. 5-336963 A (1993) or WO94/23021, the protein of the present invention is produced as a fusion protein with protein A and then the protein of interest can be purified by affinity chromatography using immunoglobulin G.

Furthermore, the protein of the present invention is produced as a fusion protein with a Flag peptide and then the protein can also be purified by affinity chromatography using an anti-Flag antibody [Proc. Natl. Acad. Sci., U.S.A., 86, 8227 (1989), Genes Develop., 4, 1288 (1990)]. Alternatively, the protein is produced as a fusion protein with polyhistidine and then can be purified by affinity chromatography using a metal coordination resin having high affinity for polyhistidine. Moreover, the protein can also be purified by affinity chromatography using an antibody against the protein itself.

Based on the amino acid sequence information of the above-obtained protein, the protein of the present invention can be produced by a chemical synthesis method such as a Fmoc method (fluorenylmethyloxycarbonyl method), a tBoc method (t-butyloxycarbonyl method), or the like. Also, the protein can also be chemically synthesized using a peptide synthesizer of Advanced ChemTech, Perkin Elmer Co., Ltd., Pharmacia, Protein Technology Instrument, Synthecell-Vega, Applied Biosystems, Shimadzu Corporation, or the like.

### 7. Process for Producing a Peptide of the Present Invention

The culture or the treated culture of a microorganism in 3 above or a transformant in 4 above, or the protein of the present invention in 1 above is used as an enzyme source. The enzyme source and one or more kinds of substrates selected from among amino acids, amino acid derivatives, and peptides are allowed to be present in an aqueous medium. A peptide is formed and accumulated in the medium, and then the peptide is recovered from the medium, so that the peptide can be produced.

### (1) Process for Producing a Peptide Using the Protein of the Present Invention as an Enzyme Source

When the protein of the present invention is used as an enzyme source in the process of the present invention, any combinations of any substrates may be used as long as one or more kinds of substrates to be used herein are amino acids selected from the group consisting of L-amino acid, Gly, and β-alanine (β-Ala), derivatives thereof, or peptides comprising the amino acids or the derivatives thereof.

Examples of L-amino acids include L-Ala, L-Gln, L-Glu, L-Val, L-Leu, L-Ile, L-Pro, L-Phe, L-Trp, L-Met, L-Ser, L-Thr, L-Cys, L-Asn, L-Tyr, L-Lys, L-Arg, L-His, L-Asp, L-citrulline (L-Cit), L-ornithine (L-Orn), L-4-hydroxyproline (L-4-HYP), L-3-hydroxyproline (L-3-HYP), L-α-aminobutyric acid (L-α-AB), L-6-diazo-5-oxo-norleucine (L-DON), L-Azaserine, and L-Theanine.

Examples of amino acid derivatives include ester compounds such as a methyl ester compound or an ethyl ester compound, an amide compound, and derivative compounds such as hydroxamate of the above L-amino acids, Gly, and β-Ala.

An example of peptides is a peptide wherein an L-amino acid, Gly, or β-Ala is linked in an arbitrary combination via α-peptide bonds.

Also in the above process for producing a peptide, a peptide, in which one or more kinds of amino acids selected from the group consisting of L-amino acid(s), Gly, and β-Ala or derivative(s) thereof are linked via L-α-peptide bonds, can be used as a substrate.

A peptide to be used as a substrate is preferably a peptide in which 1 or more kinds of 2 to 10 amino acids (selected from among L-amino acids and Gly) are linked and more preferably a peptide in which 1 or more kinds of 2 to 5 amino acids (selected from among L-amino acids and Gly) are linked. Even more preferable examples of such peptide include a peptide in which 1 or more kinds of 2 to 5 L-amino acids or Gly are linked and a dipeptide in which 2 kinds of amino acids selected from among L-amino acids and Gly are linked.

Examples of a substrate to be used in the above process include: preferably, L-Ala, L-Gln, L-Glu, L-Val, L-Leu, L-Ile, L-Pro, L-Phe, L-Trp, L-Met, L-Ser, L-Thr, L-Cys, L-Asn, L-Tyr, L-Lys, L-Arg, L-His, L-Asp, Gly, and β-Ala; one kind of amino acid selected from the group consisting of a methyl ester compound of such amino acids and hydroxamate of such amino acids or an amino acid derivative thereof; and a combination of 2 or more kinds of amino acids or amino acid derivatives. Further preferable examples of the same include: a combination of 1 or 2 kinds of amino acids or of 1 kind of amino acid and 1 kind of amino acid derivative from among amino acids selected from the group consisting of L-Ala, L-Gln, L-Glu, L-Val, L-Leu, L-Ile, L-Pro, L-Phe, L-Trp, L-Met, L-Ser, L-Thr, L-Cys, L-Asn, L-Tyr, L-Lys, L-Arg, L-His, L-Asp, Gly, and β-Ala; and a methyl ester compound of such amino acids and hydroxamate of such amino acids or amino acid derivatives thereof.

Further preferable examples of such substrate include: 1 or 2 kinds of amino acids selected from the group consisting of L-Val, L-Leu, L-Ile, L-Met, L-Ser, L-Cys, L-Lys, L-Pro, L-Trp, L-Phe, L-Ala, L-Asp, L-Tyr, L-Asn, L-Arg, L-Thr, L-His, and Gly; and a combination of 1 kind of amino acid selected from the group consisting of L-Val, L-Leu, L-Ile, L-Met, L-Ser, L-Cys, L-Lys, L-Pro, L-Trp, L-Phe, L-Ala, L-Asp, L-Tyr, L-Asn, L-Arg, L-Thr, L-His, and Gly, and 1 kind of amino acid derivative selected from the group consisting of L-arginine hydroxamate (L-ArgHx), L-valine methylester (L-ValOMe), L-LeuOMe, L-IleOMe, and L-PheOMe.

Particularly preferable examples of such substrate include: 1 kind of or a combination of 2 kinds of substrates selected from the group A when a protein having the amino acid sequence shown by SEQ ID NO: 2 is used as an enzyme source; 1 kind of or a combination of 2 kinds of substrates selected from the group B when a protein having the amino acid sequence shown by SEQ ID NO: 4 is used as an enzyme source; 1 kind of or a combination of 2 kinds of substrates selected from the group C when a protein having the amino acid sequence shown by SEQ ID NO: 6 is used as an enzyme source; 1 kind of or a combination of 2 kinds of substrates selected from the group D when a protein having the amino acid sequence shown by SEQ ID NO: 8 is used as an enzyme source; 1 kind of or a combination of 2 kinds of substrates selected from the group E when a protein having the amino acid sequence shown by SEQ ID NO: 10 is used as an enzyme source; and 1 kind of or a combination of 2 kinds of substrates selected from the group F when a protein having the amino acid sequence shown by SEQ ID NO: 12 is used as an enzyme source.

Group A: L-Val, L-Leu, L-Ile, L-Met, L-Cys, L-Lys, L-His, L-Arg, L-Pro, L-Phe, L-Tyr, and L-Trp, peptides in which these L-amino acids are bound, and derivatives of these L-amino acids. Preferable examples thereof: L-Val, L-Leu, L-Ile, L-Met, L-Cys, L-Lys, L-His, L-Arg, L-Pro, L-Phe, L-Tyr, and L-Trp, peptides in which 2 to 5 of these L-amino acids are bound, and derivatives of these L-amino acids. More preferable examples thereof: L-Val, L-Leu, L-Ile, L-Met, L-Cys, L-Lys, L-His, L-Arg, L-Pro, L-Phe, L-Tyr, and L-Trp, peptides in which 2 or 3 of these L-amino acids are bound, and derivatives of these L-amino acids. Further preferable examples thereof: L-Val, L-Leu, L-Ile, L-Met, L-Cys, L-Lys, L-Arg, L-Pro, and L-Phe, peptides in which 2 or 3 of these L-amino acids are bound, and a hydroxyamine compound and a methyl ester compound of these L-amino acids. Particularly preferable examples thereof: L-Val, L-Leu, L-Ile, and L-Met, peptides in which 2 or 3 of these L-amino acids are bound, and L-ArgHx, L-ValOMe, and L-PheOMe

Group B: L-Val, L-Leu, L-Ile, L-Met, L-Cys, L-Lys, L-His, L-Arg, L-Pro, L-Phe, L-Tyr, and L-Trp, peptides in which these L-amino acids are bound, and derivatives of these L-amino acids. Preferable examples thereof: L-Val, L-Leu, L-Ile, L-Met, L-Cys, L-Lys, L-His, L-Arg, L-Pro, L-Phe, L-Tyr, and L-Trp, peptides in which 2 to 5 of these L-amino acids are bound, and derivatives of these L-amino acids. More preferable examples thereof: L-Val, L-Leu, L-Ile, L-Met, L-Cys, L-Lys, L-His, L-Arg, L-Pro, L-Phe, L-Tyr, and L-Trp, peptides in which 2 or 3 of these L-amino acids are bound, and derivatives of these L-amino acids. Further preferable examples thereof: L-Val, L-Leu, L-Ile, L-Met, L-Cys, L-Lys, L-Arg, L-Pro, and L-Phe, peptides in which 2 or 3 of these L-amino acids are bound, and a hydroxyamine compound and a methyl ester compound of these L-amino acids. Particularly preferable examples thereof: L-Val, L-Leu, L-Ile, and L-Met, peptides in which 2 or 3 of these L-amino acids are bound, and L-ArgHx.

Group C: L-Val, L-Leu, L-Ile, L-Met, L-Cys, L-Trp, L-Phe, L-Tyr, L-Arg, L-Lys, and L-His, peptides in which these L-amino acids are bound, and derivatives of these L-amino acids. Preferable examples thereof: L-Val, L-Leu, L-Ile, L-Met, L-Cys, L-Trp, L-Phe, L-Tyr, L-Arg, L-Lys, and L-His, peptides in which 2 to 5 of these L-amino acids are bound, and derivatives of these L-amino acids. More preferable examples thereof: L-Val, L-Leu, L-Ile, L-Met, L-Cys, L-Trp, L-Phe, L-Tyr, L-Arg, L-Lys, and L-His, peptides in which 2 or 3 of these L-amino acids are bound, and derivatives of these L-amino acids. Further preferable examples thereof: L-Val, L-Leu, L-Ile, L-Met, L-Cys, L-Trp, L-Phe, and L-Arg, peptides in which 2 or 3 of these L-amino acids are bound, and a hydroxyamine compound and a methyl ester compound of these L-amino acids. Particularly preferable examples thereof: L-Val, L-Leu, L-Ile, L-Met, L-Trp, L-Phe, and L-Arg, peptides in which 2 or 3 of these L-amino acids are bound, and L-ArgHx

Group D: L-Val, L-Leu, L-Ile, L-Met, L-Cys, L-Trp, L-Phe, L-Tyr, L-Arg, L-Lys, L-His, L-Pro, L-Ser, Gly, L-Thr, L-Ala, and L-Asn, peptides in which these L-amino acids or Gly are bound, and derivatives of these L-amino acids or Gly. Preferable examples thereof: L-Val, L-Leu, L-Ile, L-Met, L-Cys, L-Trp, L-Phe, L-Tyr, L-Arg, L-Lys, L-His, L-Pro, L-Ser, Gly, L-Thr, L-Ala, and L-Asn, peptides in which 2 to 5 of these L-amino acids or Gly are bound, and derivatives of these L-amino acids or Gly. More preferable examples thereof: L-Val, L-Leu, L-Ile, L-Met, L-Cys, L-Trp, L-Phe, L-Tyr, L-Arg, L-Lys, L-His, L-Pro, L-Ser, Gly, L-Thr, L-Ala, and L-Asn, peptides in which 2 or 3 of these L-amino acids or Gly are bound, and derivatives of these L-amino acids or Gly. Further preferable examples thereof: L-Val, L-Leu, L-Ile, L-Met, L-Cys, L-Trp, L-Phe, L-Tyr, L-Arg, L-Pro, L-Ser, Gly, L-Ala, and L-Asn, peptides in which 2 or 3 of these L-amino acids or Gly are bound, and a hydroxyamine compound and a methyl ester compound of these L-amino acids or Gly. Particularly preferable examples thereof: L-Val, L-Leu, L-Ile, L-Met, L-Trp, L-Phe, L-Tyr, L-Arg, L-Pro, L-Ser, Gly, L-Ala, and L-Asn, peptides in which these L-amino acids or Gly are bound, and L-ArgHx, L-LeuOMe, and L-PheOMe

Group E: L-Val, L-Leu, L-Ile, L-Met, L-Cys, L-Trp, L-Phe, L-Tyr, L-Ala, L-Ser, L-Thr, L-Asn, and Gly, peptides in which these L-amino acids or Gly are bound and derivatives of these L-amino acids or Gly. Preferable examples thereof: L-Val, L-Leu, L-Ile, L-Met, L-Cys, L-Trp, L-Phe, L-Tyr, L-Ala, L-Ser, L-Thr, L-Asn, and Gly, peptides in which 2 to 5 of these L-amino acids or Gly are bound, and derivatives of these L-amino acids or Gly. More preferable examples thereof: L-Val, L-Leu, L-Ile, L-Met, L-Cys, L-Trp, L-Phe, L-Tyr, L-Ala, L-Ser, L-Asn, and Gly, peptides in which 2 or 3 of these L-amino acids or Gly are bound, and derivatives of these L-amino acids or Gly. Further preferable examples thereof: L-Val, L-Leu, L-Ile, L-Met, L-Cys, L-Trp, L-Phe, L-Tyr, L-Ala, L-Ser, L-Thr, L-Asn, and Gly, peptides in which 2 or 3 of these L-amino acids or Gly are bound, and a hydroxyamine compound and a methyl ester compound of these L-amino acids or Gly. Particularly preferable examples thereof: L-Val, L-Leu, L-Ile, L-Met, L-Trp, L-Tyr, L-Ala, L-Ser, L-Asn, and Gly and peptides in which 2 or 3 of these L-amino acids or Gly are bound.

Group F: L-Val, L-Leu, L-Ile, L-Met, L-Cys, L-Trp, L-Phe, L-Tyr, L-Asp, L-Ser, Gly, L-Thr, L-Arg, L-His, and L-Lys, peptides in which these L-amino acids or Gly are bound and derivatives of these L-amino acids or Gly. Preferable examples thereof: L-Val, L-Leu, L-Ile, L-Met, L-Cys, L-Trp, L-Phe, L-Tyr, L-Asp, L-Ser, Gly, L-Thr, L-Arg, L-His, and L-Lys, peptides in which 2 to 5 of these L-amino acids or Gly are bound, and derivatives of these L-amino acids or Gly. More preferable examples thereof: L-Val, L-Leu, L-Ile, L-Met, L-Cys, L-Trp, L-Phe, L-Tyr, L-Asp, L-Ser, Gly, L-Thr, L-Arg, L-His, and L-Lys, peptides in which 2 or 3 of these L-amino acids or Gly are bound, and derivatives of these L-amino acids or Gly. Further preferable examples thereof: L-Val, L-Leu, L-Ile, L-Met, L-Cys, L-Trp, L-Phe, L-Tyr, L-Asp, L-Ser, Gly, L-Thr, L-Arg, L-His, and L-Lys, peptides in which 2 or 3 of these L-amino acids or Gly are bound, and a hydroxyamine compound and a methyl ester compound of these L-amino acids or Gly. Particularly preferable examples thereof: L-Val, L-Leu, L-Ile, L-Met, L-Cys, L-Trp, L-Phe, L-Tyr, L-Asp, L-Ser, L-Thr, L-Arg, and L-His, peptides in which 2 or 3 of these L-amino acids are bound, and L-PheOMe and L-LeuOMe

In the above process, 0.01 mg to 100 mg, preferably 0.1 mg to 10 mg of the protein of the present invention is added per mg of amino acid to be used as a substrate.

In the above process, an amino acid to be used as a substrate is added initially or during the reaction to an aqueous medium at a concentration ranging from 0.1 g/L to 500 g/L, preferably ranging from 0.2 g/L to 200 g/L.

In the above process, ATP can be used as an energy source at a concentration ranging from 0.5 mmol to 10 mol/L.

An aqueous medium to be used in the above process may have any ingredients or composition, as long as a reaction for peptide production is not inhibited. Examples of such aqueous medium include water, a phosphate buffer, a carbonate buffer, an acetate buffer, a borate buffer, a citrate buffer, and a tris buffer. Also, such an aqueous medium may contain alcohols such as methanol and ethanol, esters such as ethyl acetate, ketones such as acetone, and amides such as acetamide.

The reaction for peptide production is carried out in an aqueous medium under conditions of pH 5 to 11 and preferably pH6 to 10, at 20° C. to 50° C. and preferably 25° C. to 45° C., and for 2 to 150 hours and preferably 6 to 120 hours.

Examples of amino acids, amino acid derivatives, and peptides composing peptides produced by the above processes include amino acids, amino acid derivatives, and peptides to be used as substrates in the above process.

Also, examples of components of peptides that are produced by the above processes include: preferably a substrate or an amino acid derivative of the substrate selected from the group A above when a protein having the amino acid sequence shown by SEQ ID NO: 2 is used as an enzyme source; preferably a substrate or an amino acid derivative of the substrate selected from the group B above when a protein having the amino acid sequence shown by SEQ ID NO: 4 is used as an enzyme source; preferably a substrate or an amino acid derivative of the substrate selected from the group C above when a protein having the amino acid sequence shown by SEQ ID NO: 6 is used as an enzyme source; preferably a substrate or an amino acid derivative of the substrate selected from the group D above when a protein having the amino acid sequence shown by SEQ ID NO: 8 is used as an enzyme source; preferably a substrate or an amino acid derivative of the substrate selected from the group E above when a protein having the amino acid sequence shown by SEQ ID NO: 10 is used as an enzyme source; and preferably a substrate or an amino acid derivative of the substrate selected from the group F above when a protein having the amino acid sequence shown by SEQ ID NO: 12 is used as an enzyme source.

Regarding the length of a peptide to be produced by the above processes, an example of such peptide is a peptide, in which 2 to 20, preferably 2 to 10, more preferably 2 to 8, and further preferably 2 to 6 substrates that are of one or more kinds selected from among amino acids and amino acid derivatives are bound via peptide bonds.

Examples of a peptide to be produced by the above processes include a peptide in which 1 or more kinds of substrates selected from among amino acids and amino acid derivatives are bound via peptide bonds, preferably a peptide in which 1 or more kinds of amino acids selected from among L-amino acids and Gly are bound via peptide bonds, and a peptide in which an amino acid derivative is bound to the C-terminus of the peptide, more preferably a peptide in which 1 kind of amino acid selected from among L-amino acids and Gly is bound via peptide bonds, and a peptide in which an amino acid derivative is bound to the C-terminus of the peptide.

Moreover, examples of a peptide to be produced by the above processes include:

i) when a protein having the amino acid sequence shown by SEQ ID NO: 2 is used as an enzyme source, preferably a peptide in which 1 or more kinds of L-amino acids selected from among L-Val, L-Leu, L-Ile, L-Met, L-Cys, L-Lys, L-His, L-Arg, L-Pro, L-Phe, L-Tyr, and L-Trp are bound or a derivative of the peptide, more preferably a peptide in which 1 or more kinds of L-amino acids selected from among L-Val, L-Leu, L-Ile, L-Met, L-Cys, L-Lys, L-His, L-Arg, L-Pro, L-Phe, L-Tyr, and L-Trp are bound or a hydroxyamine compound or a methyl ester compound of the peptide, further preferably a peptide in which 1 or more kinds of L-amino acids selected from among L-Val, L-Leu, L-Ile, L-Met, L-Cys, L-Lys, L-Arg, L-Pro, and L-Phe are bound or a hyroxyamine compound or a methyl ester compound of the peptide, and particularly preferably a peptide in which 1 or more kinds of L-amino acids selected from among L-Val, L-Leu, L-Ile, L-Met, L-Arg, and L-Phe are bound or a hydroxyamine compound or a methyl ester compound of the peptide;

ii) when a protein having the amino acid sequence shown by SEQ ID NO: 4 is used as an enzyme source, preferably a peptide in which 1 or more kinds of L-amino acids selected from among L-Val, L-Leu, L-Ile, L-Met, L-Cys, L-Lys, L-Arg, L-His, L-Pro, L-Phe, L-Tyr, and L-Trp are bound or a derivative of the peptide, more preferably a peptide in which 1 or more kinds of L-amino acids selected from among L-Val, L-Leu, L-Ile, L-Met, L-Cys, L-Lys, L-Arg, L-His , L-Pro, L-Phe, L-Tyr, and L-Trp are bound or a hydroxyamine compound or a methyl ester compound of the peptide, further preferably a peptide in which 1 or more kinds of L-amino acids selected from among L-Val, L-Leu, L-Ile, L-Met, L-Cys, L-Lys, L-Pro, and L-Arg are bound or a hydroxyamine compound or a methyl ester compound of the peptide, and particularly preferably a peptide in which 1 or more kinds of L-amino acids selected from among L-Val, L-Leu, L-Ile, L-Met, and L-Arg are bound and a hydroxyamine compound or a methyl ester compound of the peptide;

iii) when a protein having the amino acid sequence shown by SEQ ID NO: 6 is used as an enzyme source, preferably a peptide in which 1 or more kinds of L-amino acids selected from among L-Val, L-Leu, L-Ile, L-Met, L-Cys, L-Trp, L-Phe, L-Tyr, L-Arg, L-Lys, and L-His are bound or a derivative of the peptide, more preferably a peptide in which 1 or more kinds of L-amino acids selected from among L-Val, L-Leu, L-Ile, L-Met, L-Cys, L-Trp, L-Phe, L-Tyr, L-Arg, L-Lys, and L-His are bound or a hydroxyamine compound or a methyl ester compound of the peptide, further preferably a peptide in which 1 or more kinds of L-amino acids selected from among L-Val, L-Leu, L-Ile, L-Met, L-Cys, L-Trp, L-Phe, and L-Arg are bound or a hydroxyamine compound or a methyl ester compound of the peptide, and particularly preferably a peptide in which 1 or more kinds of L-amino acids selected from among L-Val, L-Leu, L-Ile, L-Met, L-Trp, L-Phe, and L-Arg are bound or a hydroxyamine compound or a methyl ester compound of the peptide;

iv) when a protein having the amino acid sequence shown by SEQ ID NO: 8 is used as an enzyme source, preferably a peptide in which 1 or more kinds of L-amino acids selected from among L-Val, L-Leu, L-Ile, L-Met, L-Cys, L-Trp, L-Phe, L-Tyr, L-Arg, L-Lys, L-His, L-Pro, L-Ser, L-Thr, L-Ala, and L-Asn or Gly are bound or a derivative of the peptide, more preferably a peptide in which 1 or more kinds of L-amino acids selected from among L-Val, L-Leu, L-Ile, L-Met, L-Cys, L-Trp, L-Phe, L-Tyr, L-Arg, L-Lys, L-His, L-Pro, L-Ser, L-Thr, L-Ala, and L-Asn or Gly are bound or a hydroxyamine compound or a methyl ester compound of the peptide, further preferably a peptide in which 1 or more kinds of L-amino acids selected from among L-Val, L-Leu, L-Ile, L-Met, L-Cys, L-Trp, L-Phe, L-Tyr, L-Arg, L-Pro, L-Ser, L-Ala, and L-Asn or Gly are bound or a hydroxyamine compound or a methyl ester compound of the peptide, and particularly preferably a peptide in which 1 or more kinds of L-amino acids selected from among L-Val, L-Leu, L-Ile, L-Met, L-Trp, L-Phe, L-Tyr, L-Arg, L-Pro, L-Ser, L-Ala, and L-Asn or Gly are bound or a hydroxyamine compound or a methyl ester compound of the peptide;

v) when a protein having the amino acid sequence shown by SEQ ID NO: 10 is used as an enzyme source, preferably a peptide in which 1 or more kinds of L-amino acids selected from among L-Val, L-Leu, L-Ile, L-Met, L-Cys, L-Trp, L-Phe, L-Tyr, L-Ala, L-Ser, L-Thr, and L-Asn or Gly are bound or a derivative of the peptide, more preferably a peptide in which 1 or more kinds of L-amino acids selected from among L-Val, L-Leu, L-Ile, L-Met, L-Cys, L-Trp, L-Phe, L-Tyr, L-Ala, L-Ser, L-Thr, and L-Asn or Gly are bound or a hydroxyamine compound or a methyl ester compound of the peptide, further preferably a peptide in which 1 or more kinds of L-amino acids selected from among L-Val, L-Leu, L-Ile, L-Met, L-Cys, L-Trp, L-Phe, L-Tyr, L-Ala, L-Ser, and L-Asn or Gly are bound or a hydroxyamine compound or a methyl ester compound of the peptide, and particularly preferably a peptide in which 1 or more kinds of L-amino acids selected from among L-Val, L-Leu, L-Ile, L-Met, L-Trp, L-Phe, L-Tyr, L-Ala, L-Ser, and L-Asn or Gly are bound or a hydroxyamine compound or a methyl ester compound of the peptide; and

vi) when a protein having the amino acid sequence shown by SEQ ID NO: 12 is used as an enzyme source, preferably a peptide in which 1 or more kinds of L-amino acids selected from among L-Val, L-Leu, L-Ile, L-Met, L-Cys, L-Trp, L-Phe, L-Tyr, L-Asp, L-Ser, L-Thr, L-Arg, L-His, and L-Lys or Gly are bound or a derivative of the peptide, more preferably a peptide in which 1 or more kinds of L-amino acids selected from among L-Val, L-Leu, L-Ile, L-Met, L-Cys, L-Trp, L-Phe, L-Tyr, L-Asp, L-Ser, L-Thr, L-Arg, L-His, and L-Lys or Gly are bound or a hydroxyamine compound or a methyl ester compound of the peptide, further preferably a peptide in which 1 or more kinds of L-amino acids selected from among L-Val, L-Leu, L-Ile, L-Met, L-Cys, L-Trp, L-Phe, L-Tyr, L-Asp, L-Ser, L-Thr, L-Arg, and L-His are bound or a hydroxyamine compound or a methyl ester compound of the peptide, and particularly preferably a peptide in which 1 or more kinds of L-amino acids selected from among L-Val, L-Leu, L-Ile, L-Met, L-Cys, L-Trp, L-Phe, L-Tyr, L-Asp, L-Ser, L-Thr, L-Arg, and L-His are bound or a hydroxyamine compound or a methyl ester compound of the peptide.

### (2) Process for Producing a Peptide Using the Culture or the Treated Culture of a Microorganism or a Transformant as an Enzyme Source

An example of the culture of a microorganism or a transformant, which is used as an enzyme source in the process of the present invention, is a culture that is obtained by culturing the microorganism or the transformant by the culturing method in 6 above. Examples of the treated culture of a microorganism or a transformant include those containing living cells retaining functions, as enzyme sources, similar to those of the culture, such as a concentrated culture, a dried culture, cells obtained by centrifugation, filtration, or the like of the culture, a dried cell, a freeze-dried cell, a cell treated with a surfactant, a cell treated with a solvent, a cell treated with an enzyme, and an immobilized cell.

When the culture or the treated culture of a microorganism or a transformant is used as an enzyme source, examples of one or more kinds of amino acid to be used as a substrate is an amino acid(s) or derivative(s) thereof similar to that (those) in (1) above.

The amount of the enzyme source differs depending on the specific activity or the like of the enzyme source. For example, 5 mg to 1000 mg, preferably 10 mg to 400 mg as wet cell weight of the enzyme source is added per mg of amino acid to be used as a substrate.

An amino acid or a derivative thereof to be used as a substrate can be added into an aqueous medium in a manner similar to that in (1) above.

ATP is allowed to be present in an aqueous medium in a manner similar to that in (1) above, so that ATP can be used as an energy source.

Also in the above processes, if necessary, ATP or a compound with which ATP can be produced via metabolization of a transformant or a microorganism, such as saccharides (e.g., glucose), alcohols (e.g., ethanol), or organic acids (e.g., acetic acid) can be added as an ATP supply source to an aqueous medium.

Media in (1) above can be used as aqueous media. In addition, a supernatant of the culture of a microorganism or a transformant, which is used as an enzyme source, can also be used as an aqueous medium.

Also in the above process, a surfactant or an organic solvent may also be added into an aqueous medium, as necessary. Any surfactant may be used herein, as long as it accelerates the production of galactose-containing complex carbohydrate. Examples of such surfactant include nonionic surfactants such as polyoxyethylene octadecylamine (e.g., NYMEEN S-215, NOF Corporation), cationic surfactants such as cetyltrimethylammonium bromide and alkyldimethylbenzylammonium chloride (e.g., cation F2-40E, NOF Corporation), anionic surfactants such as lauroyl sarcosinate, and tertiary amines such as alkyldimethylamine (e.g., tertiary amine FB, NOF Corporation). One kind or several kinds thereof can also be mixed and then used. Such a surfactant is generally used at a concentration ranging from 0.1 g/l to 50 g/l. Examples of an organic solvent include xylene, toluene, aliphatic alcohol, acetone, and ethyl acetate and such an organic solvent is generally used at a concentration ranging from 0.1 ml/l to 50 ml/l.

The reaction conditions for a reaction for peptide production are the same conditions as those in (1) above, for example.

An example of a peptide to be produced by the above processes is the same peptide as that in (1) above.

(3) Process for Producing a Peptide Using a Microorganism that has an Ability to Produce a Protein Having Peptide-Synthesizing Activity and has an Ability to Produce at Least One Kind of Substrate Selected from Among Amino Acids, Amino Acid Derivatives, and Peptides

A process for producing a peptide, which is characterized by culturing a microorganism that has an ability to produce a protein having peptide-synthesizing activity and has an ability to produce at least one kind of substrate selected from among amino acids, amino acid derivatives, and peptides in a medium, allowing peptides to form and accumulate in the medium, and then recovering peptides from the medium is also an example of the process of the present invention.

Examples of such microorganism that has an ability to produce a protein having peptide-synthesizing activity and has an ability to produce at least one kind of substrate selected from among amino acids, amino acid derivatives, and peptides include a microorganism obtained by transformation of a microorganism having an ability to produce an amino acid, an amino acid derivative or a peptide to be used as a substrate in the process for producing a peptide of the present invention, preferably an L-amino acid or Gly or a peptide, or more preferably an L-amino acid or Gly with the DNA of the present invention.

Examples of such microorganism having an ability to produce an amino acid, an amino acid derivative, or a peptide include a strain itself when the strain isolated from nature has such ability of its own and a microorganism to which the ability to produce a desired substrate is artificially given by a known method.

Examples of such known method for artificially giving the ability to produce an amino acid to a microorganism include:
(a) a method in which at least one of the mecahnisms regulating the biosynthesis of the amino acid is relaxed or canceled;
(b) a method in which the expression of at least one of the enzymes involved in the biosynthesis of the amino acids is enhanced;
(c) a method in which the copy number of at least one of the enzyme genes involved in the biosynthesis of the amino acid is increased;
(d) a method in which at least one of the metabolic pathways branching from the biosynthestic pathway of the amino acid into metabolites other than the amino acid is weakened or blocked; and
(e) a method in which a cell strain having a higher resistance to an analogue of the amino acid as compared with a wild-type strain is selected. The above known methods can be used independently or in combination.

The above method (a) is described in Agric. Biol. Chem., 43, 105-111(1979), J. Bacteriol., 110, 761-763 (1972) and Appl. Microbiol. Biotechnol., 39, 318-323 (1993), for example. The above method (b) is described in Agric. Biol. Chem., 43, 105-111 (1979) and J. Bacteriol., 110, 761-763(1972), for example. The above method (c) is described in Appl. Microbiol. Biotechnol., 39, 318-323 (1993) and Agric. Biol. Chem., 39, 371-377(1987), for example. The above method (d) is described in Appl. Environ. Microbiol., 38, 181-190 (1979) and Agric. Biol. Chem., 42, 1773-1778 (1978), for example. The above method (e) is described in Agric. Biol. Chem., 36, 1675-1684 (1972), Agric. Biol. Chem., 41, 109-116 (1977), Agric. Biol. Chem., 37, 2013-2023 (1973), and Agric. Biol. Chem., 51, 2089-2094(1987), for example. Microorganisms which have an ability to produce various amino acids can be prepared in reference to the above documents and the like.

Regarding methods for preparing microorganisms having an ability to produce amino acids using any one of or a combination of the above methods (a) to (e), many examples thereof have been described in Biotechnology 2nd ed., Vol. 6, Products of Primary Metabolism (VCH Verlagsgesellschaft mbH, Weinheim, 1996) section 14a, 14b and Advances in Biochemical Engineering/Biotechnology 79, 1-35 (2003), Amino Acid Fermentation, Japan Scientific Societies Press, Hiroshi Aida et al., (1986). In addition to the above, there are many reports concerning specific methods for preparing microorganisms having an ability to produce amino acids, including JP Patent Publication (Kokai) No. 2003-164297 A, Agric. Biol. Chem., 39, 153-160 (1975), Agric. Biol. Chem., 39, 1149-1153 (1975), JP Patent Publication (Kokai) No. 58-13599 A (1983), J. Gen. Appl. Microbiol., 4, 272-283 (1958), JP Patent Publication (Kokai) No. 63-94985 A (1988), Agric. Biol. Chem., 37, 2013-2023 (1973), WO97/15673, JP Patent Publication (Kokai) No. 56-18596 A (1981), JP Patent Publication (Kokai) No. 56-144092 A (1981), JP Patent Publication (Kohyo) No. 2003-511086 A, and the like. Microorganisms having an ability to produce amino acids can be prepared in reference to the above documents and the like.

Specific examples of microorganisms producing amino acids include: Escherichia coli JGLE1 and Escherichia coli JGLBE1 as L-glutamine-producing strains; Escherichia coli JM101 strain and the like retaining an ald gene expression plasmid as L-alanine-producing strains; Escherichia coli JM101 strain and the like retaining a pPHEA2 and/or aroF gene expression plasmid as L-phenylalanine-producing strains; Escherichia coli JGLE1, Escherichia coli JGLBE1, and the like retaining an ald gene expression plasmid as L-glutamine- and L-alanine-producing strains; Escherichia coli JM101 and the like retaining an ald gene expression plasmid and a desensitization pheA gene and/or a desensitization aroF gene expression plasmid as L-alanine- and L-phenylalanine-producing strains; and ATCC21277 strain and the like retaining a desensitization pheA gene and/or a desensitization aroF gene expression plasmid as L-threonine- and L-phenylalanine-producing strains.

Furthermore, specific examples of microorganisms having an ability to produce amino acids include: FERM BP-5807 and ATCC13032 as L-glutamic acid-producing strains; FERM P-4806 and ATCC14751 as L-glutamine-producing strains; ATCC21148, ATCC21277, and ATCC21650 as L-threonine-producing strains; FERM P-5084 and ATCC13286 as L-lysine-producing strains; FERM P-5479, VKPM B-2175, and ATCC21608 as L-methionine-producing strains; FERM BP-3757 and ATCC14310 as L-isoleucine-producing strains; ATCC13005 and ATCC19561 as L-valine-producing strains; FERM BP-4704 and ATCC21302 as L-leucine-producing strains; FERM BP-4121 and ATCC15108 as L-alanine-producing strains; ATCC21523 and FERM BP-6576 as L-serine-producing strains; FERM BP-2807 and ATCC 19224 as L-proline-producing strains; FERM P-5616 and ATCC21831 as L-arginine-producing strains; ATCC13232 and the like as L-ornithine-producing strains; FERM BP-6674 and ATCC21607 as L-histidine-producing strains; DSM10118, DSM10121, DSM10123, and FERM BP-1777 as L-tryptophan-producing strains; ATCC13281 and ATCC21669 as L-phenylalanine-producing strains; ATCC21652 and the like as L-tyrosine-producing strains; W3110/pHC34 (described in JP Patent Publication (Kohyo) No. 2003-511086 A) and the like as L-cysteine-producing strains; Escherichia coli SOLR/pRH71 and the like (described in WO96/27669) as L-4-hydroxyproline-producing strains; FERM BP-5026 and FERM BP-5409 as L-3-hydroxyproline-producing strains; and FERM P-5643 and FERM P-1645 as L-citrulline-producing strains.

In addition, microbial strains identified by the above FERM Nos. are available from the International Patent Organism Depositary, National Institute of Advanced Industrial Science and Technology (Japan); microbial strains identified by ATCC Nos. are available from the American Type Culture Collection (U.S.A.); microbial strains identified by VKPM Nos. are available from the Russian National Collection of Industrial Microorganisms (Russia); and microbial strains identified by DSM Nos. are available from Deutsche Sammlung von Mikroorganismen and Zellkulturen (Germany).

An example of a microorganism having an ability to produce a peptide is a microorganism having an ability to produce a dipeptide as described in WO2006/001379, for example.

Examples of a method for transforming the above microorganisms having an ability to produce amino acids, amino acid derivatives, or peptides with the DNA of the present invention include methods similar to the process for producing transformants of the present invention as described in 5 above.

Such microorganism obtained as described above, which has an ability to produce a protein that has peptide-synthesizing activity and has an ability to produce at least one kind of substrate selected from among amino acids, amino acid derivatives, and peptides, can be cultured in a manner similar to that in the methods described in 6 above.

In the processes of (1) to (3) above, peptides formed and accumulated in aqueous media are recovered by a general method using activated carbon, ion exchange resin, or the like or extraction using an organic solvent, crystallization, thin-layer chromatography, high-performance liquid chromatography (HPLC), or the like.

Examples of the present invention are as described below. Escherichia coli JPNDABPOPepT1 strain used in the Examples was prepared by the following method.

Microbial strains in which pepD gene, pepN gene, pepA gene, pepB gene, pepT gene, dpp operon, and opp operon on the chromosomal DNA of the Escherichia coli JM101 strain are deleted were prepared according to the method using a homologous recombination system of a lambda phage [Proc. Natl. Acad. Sci. U.S.A., 97, 6641-6645 (2000)].

Plasmids pKD46, pKD3, and pCP20 used herein were prepared by obtaining Escherichia coli strains retaining the plasmids from the Escherichia coli Genetic Stock Center (Yale University (U.S.A.)) and then extracting the plasmids by a known method from the strains.

### (1) Cloning of DNA Fragments for Gene Deletion

A DNA comprising the nucleotide sequences shown by SEQ ID NOS: 27 and 28 was used as a primer set for amplification of a DNA fragment to be used for pepD gene deletion. A DNA comprising the nucleotide sequences shown by SEQ ID NOS: 29 and 30 was used as a primer set for amplification of a DNA fragment to be used for pepN gene deletion. A DNA comprising the nucleotide sequences shown by SEQ ID NOS: 31 and 32 was used as a primer set for amplification of a DNA fragment to be used for pepA gene deletion. A DNA comprising the nucleotide sequences shown by SEQ ID NOS: 33 and 34 was used as a primer set for amplification of a DNA fragment to be used for pepB gene deletion. A DNA comprising the nucleotide sequences shown by SEQ ID NO: 35 and 36 was used as a primer set for amplification of a DNA fragment to be used for pepT gene deletion. A DNA comprising the nucleotide sequences shown by SEQ ID NO: 37 and 38 was used as a primer set for amplification of a DNA fragment to be used for dpp operon deletion. Furthermore, a DNA comprising the nucleotide sequences shown by SEQ ID NO: 39 and 40 was used as a primer set for amplification of a DNA fragment to be used for opp operon deletion. PCR was carried out using these primer sets and pKD3 as a template. PCR was carried out by repeating 30 times a step comprising 94° C. for 1 minute, 55° C. for 2 minutes, 72° C. for 3 minutes using 40 μL of a reaction solution containing 10 ng of pKD3, primers (0.5 μmol/L each), 2.5 units of Pfu DNA polymerase, 4 μL of ×10 buffer for Pfu DNA polymerase, and deoxyNTP (200 μmol/L each).

1/10 the amount of each reaction solution was subjected to agarose gel electrophoresis, so as to confirm amplification of a target fragment. TE-saturated phenol/chloroform in an amount equivalent to that of the remaining reaction solution was added and then mixed therewith. The mixture was centrifuged, cold ethanol in an amount twice that of the thus obtained upper layer was added to and mixed with the upper layer, and then the solution was left to stand at −80° C. for 30 minutes. The solution was centrifuged so that a DNA fragment containing a chloramphenicol resistance gene for pepD gene, pepN gene, pepA gene, pepB gene, pepT gene, dpp operon, and opp operon deletion was obtained.

(2) Preparation of pepD Gene-Deleted Escherichia coli JM101

After transformation of the Escherichia coli JM101 strain with a plasmid pKD46, the strain was spread on LB agar medium containing 100 mg/L ampicillin. After culturing at 30° C., the Escherichia coli JM101 strain (hereinafter, referred to as Escherichia coli JM101/pKD46) retaining pKD46 was selected.

The DNA fragment for pepD gene deletion (obtained in (1) above) was introduced into Escherichia coli JM101/pKD46 obtained by culturing thereof in the presence of 10 mmol/L L-arabinose and 50 μg/ml ampicillin by an electric pulse method. The resultant was spread on LB agar medium containing 25 mg/L chloramphenicol, and then cells were cultured overnight at 30° C. A transformed cell, in which the DNA fragment for pepD gene deletion had been integrated by homologous recombination onto Escherichia coli JM101 chromosomal DNA, was selected using chloramphenicol resistance as an index.

The thus selected chloramphenicol resistant strain was inoculated on LB agar medium containing 25 mg/L chloramphenicol and then cultured at 42° C. for 14 hours. Single colonies were then isolated. Each of the thus obtained colonies was inoculated on LB agar medium containing 25 mg/L chloramphenicol and LB agar medium containing 100 mg/L ampicillin and then cultured at 37° C.

Through selection of colonies exerting chloramphenicol resistance and ampicillin sensitivity, a pKD46-eliminated strain was obtained.

Next, the above-obtained pKD46-eliminated strain was transformed with pCP20 and then an ampicillin resistant strain was selected on LB agar medium containing 100 mg/L ampicillin, so that the pKD46-eliminated strain retaining pCP20 was obtained.

The above obtained pCP20-retaining pKD46-eliminated strain was inoculated on LB agar medium to which no drug had been added and then cultured at 42° C. for 14 hours. Single colonies were then isolated. The thus obtained colonies were each inoculated on LB agar medium to which no drug had been added, LB agar medium containing 25 mg/L chloramphenicol, and LB agar medium containing 100 mg/L ampicillin. After culturing at 30° C., colonies exerting chloramphenicol sensitivity and ampicillin sensitivity were selected.

Chromosomal DNA was prepared from each strain selected above according to a conventional method.

PCR was carried out using the thus obtained chromosomal DNA as a template, a DNA having the nucleotide sequences shown by SEQ ID NOS: 41 and 42 as a primer set, a reaction solution prepared to have the composition similar to that in (1) above and a similar reaction cycle.

As a result, a strain for which no amplified DNA fragment had been detected was determined as a pepD gene-deleted strain and designated an Escherichia coli JPD1 strain.

(3) Preparation of a Strain in which pepD Gene, pepN Gene, pepA Gene, pepB Gene, pepT Gene, dpp Operon, and opp Operon on a Chromosomal DNA of Escherichia coli JM 101 are Deleted.

A strain in which pepD gene, pepN gene, pepA gene, pepB gene, pepT gene, dpp operon, and opp operon on the chromosomal DNA of Escherichia coli JM101 strain are deleted was prepared by repeating a method similar to that in (2) above using the Escherichia coli JPD1 strain obtained in (2) above and DNA fragments (obtained in (1) above) for pepN gene deletion, pepA gene deletion, pepB gene deletion, pepT gene deletion, dpp operon gene deletion, and opp operon gene deletion, respectively.

Deletion of each gene as a result of the above method was confirmed by PCR similar to that in (2) above using DNAs, as primer sets, having the nucleotide sequences shown by SEQ ID NOS: 43-54, which had been designed and synthesized based on the inner nucleotide sequence of each deleted gene. Here, a DNA comprising the nucleotide sequences shown by SEQ ID NOS: 43 and 44 was a primer set for confirmation of pepN gene deletion, a DNA comprising the nucleotide sequences shown by SEQ ID NOS: 45 and 46 was a primer set for confirmation of pepA gene deletion, a DNA comprising the nucleotide sequences shown by SEQ ID NOS: 47 and 48 was a primer set for confirmation of pepB gene deletion, a DNA comprising the nucleotide sequences shown by SEQ ID NOS: 49 and 50 was a primer set for confirmation of pepT gene deletion, a DNA comprising the nucleotide sequences shown by SEQ ID NOS: 51 and 52 was a primer set for confirmation of dpp operon deletion, and a DNA comprising the nucleotide sequences shown by SEQ ID NOS: 53 and 54 was a primer set for confirmation of opp operon deletion.

A strain obtained by the above method, in which pepD, pepN, pepA, pepB, pepT, dppA, dppB, dppC, dppD, dppF, oppA, oppB, oppC, oppD, and oppF genes were deleted was designated an Escherichia coli JPNDABPOPepT1 strain.

In the following Examples, analysis and determination by HPLC for dipeptides and amino acids were carried out by the following methods.

Dipeptides and amino acids were derivatized using Nα-1-fluoro-2,4-dinitrophenyl-5-L-alanine amide sodium (Nα-1-Fuluoro-2,4-dinitrophenyl-5-L-alanine amide, hereinafter, abbreviated as Nα-FDAA) and then subjected to HPLC analysis. Derivatization using Nα-FDAA was carried out by adding 50 μl of a 0.5% Nα-FDAA acetone solution and 40 μl of a 0.5 mol/l aqueous sodium carbonate solution to 100 μl of a sample diluted with pure water, stirring the solution, and then leaving the solution to stand at 40° C. for 60 minutes.

Next, 40 μl of 1 mol/l hydrochloric acid and 770 μl of methanol were added and then the solution was stirred, so as to prepare an FDAA sample (the sample treated with FDAA).

The FDAA sample was analyzed and determined using a WH-C18A column (Hitach, Ltd. Science Systems, 4×150 mm). Conditions employed for HPLC analysis are as follows.

As mobile phases, mobile phase A in which the mixing ratio of 50 mmol/l potassium phosphate buffer (adjusted to pH 2.7 using phosphoric acid) to acetonitrile to methanol was 18:1:1, mobile phase B in which the mixing ratio of 50 mmol/l potassium phosphate buffer (adjusted to pH 2.7 using phosphoric acid) to acetonitrile to methanol was 12:7:1, and mobile phase C in which the mixing ratio of acetonitrile to tetrahydrofuran to water was 3:1:1 were used. The flow rate of each mobile phase was 0.5 ml/min. The mixing ratio of mobile phase A to mobile phase B to mobile phase C was changed to form a gradient ranging from 100:0:0 to 55:45:0 during minutes 0 to 24, kept at 55:45:0 during minutes 24 to 30, changed to form a gradient ranging from 55:45:0 to 0:100:0 during minutes 30 to 50, kept at 0:100:0 during minutes 50 to 55, changed to form a gradient ranging from 0:100:0 to 0:0:100 during minutes 55 to 60, kept at 0:0:100 during minutes 60 to 62, changed to form a gradient ranging from 0:0:100 to 100:0:0 during minutes 62 to 62.1, and kept at 100:0:0 during minutes 62.1 to 80. The column temperature was 40° C. and ultraviolet absorption was measured at 340 nm.

### Example 1

### Construction of Strains Expressing Peptide Synthesizing Enzyme

(1) Construction of a Strain Expressing Peptide Synthesizing Enzyme from Bacillus subtilis ATCC6633 Strain

A DNA fragment having the nucleotide sequence shown by SEQ ID NO: 1 was obtained from the chromosomal DNA of the Bacillus subtilis ATCC6633 strain.

First, the Bacillus subtilis ATCC6633 strain was spread on YPGA medium [7 g/L yeast extract (Difco), 7 g/L bacto peptone (Difco), 7 g/L glucose, and 1.5 g/L agar] and statically cultured at 30° C. overnight. One platinum loop of the thus grown cells was inoculated in 3 mL of YPG medium [7 g/L yeast extract (Difco), 7 g/L bacto peptone (Difco), and 7 g/L glucose], followed by 24 hours of shaking culture at 30° C. Cells were separated by centrifugation from the thus obtained culture solution and then chromosomal DNA was prepared using a Dneasy Kit (QIAGEN) from the obtained cells.

PCR was carried out using synthetic DNAs having the nucleotide sequences shown by SEQ ID NOS: 13 and 14 as a primer set and the chromosomal DNA of the Bacillus subtilis ATCC6633 strain as a template. Specifically, PCR was carried out as follows by preparing 50 μL of a reaction solution containing 0.50 pg of chromosomal DNA, primers (0.3 μmol/L each), 1 unit of KOD plus DNA polymerase (Toyobo Co., Ltd.), 5 μL of ×10 buffer for KOD plus DNA polymerase (Toyobo Co., Ltd.), and dNTP (200 μmol/L each) (dATP, dGTP, dCTP, and dTTP) was prepared. After heating the solution at 94° C. for 120 seconds, a step of heating the solution at 94° C. for 15 seconds, 50° C. for 30 seconds, and 68° C. for 120 seconds was repeated 25 times, followed by 5 minutes of heating at 68° C.

1/10 the amount of the reaction solution was subjected to agarose gel electrophoresis, so that the amplification by PCR of an approximately 1.2-kb DNA fragment was confirmed.

Next, the remaining reaction solution was mixed with a TE [10 mmol/L Tris-HCl (pH 8.0), 1 mmol/L EDTA]-saturated phenol/chloroform (1 vol/1 vol) solution in an amount equivalent thereto. The solution was centrifuged to obtain an upper layer. Cold ethanol in an amount twice that of the thus obtained upper layer was added to and mixed with the upper layer, and then the solution was left to stand at −80° C. for 30 minutes. The solution was centrifuged to precipitate DNA and then the DNA was dissolved in 20 μL of TE.

5 μL of the solution (in which the DNA had been dissolved) was subjected to digestion with restriction enzymes Nde I and BamH I and then a DNA fragment was separated by agarose gel electrophoresis. An approximately 1.2-kb DNA fragment was collected using a GENECLEAN II kit (BIO 101).

pET-21a(+) (Novagen) (0.2 μg) was subjected to digestion with restriction enzymes Nde I and BamH I, and then a DNA fragment was separated by agarose gel electrophoresis. An approximately 5.4-kb DNA fragment was collected by a method similar to the above.

The thus obtained approximately 1.2-kb fragment and approximately 5.4-kb fragment were subjected to 16 hours of reaction at 16° C. for ligation using a ligation kit (Takara Bio Inc.).

The Escherichia coli JM109 strain was transformed with the reaction solution by a method using calcium ions [Proc. Natl. Acad. Sci., U.S.A., 69, 2110 (1972)]. The transformant was spread on LB agar medium containing 50 μg/mL ampicillin and then cultured overnight at 30° C., so that a transformed cell was selected.

The transformed cell was cultured overnight on LB medium containing 50 μg/ml ampicillin. A plasmid was prepared by an alkaline SDS method (Molecular Cloning, 3rd Edition) from the thus obtained culture solution.

Restriction enzyme digestion analysis was carried out. It was confirmed that the plasmid had a structure in which an approximately 1.2-kb DNA fragment (obtained above) had been inserted into pET-21a(+). This plasmid was designated pRBS.

Escherichia coli strain BL21 (DE3) (Novagen) was transformed with pRBS by a method using calcium ions. The transformant was spread on LB agar medium containing 50 μg/mL ampicillin and then cultured overnight at 30° C., so that a transformed cell was selected.

A plasmid was extracted by an alkaline SDS method from colonies of the transformant that had grown. The structure was analyzed using restriction enzymes, so as to confirm that pRBS was obtained.

The thus obtained transformed cell was designated Escherichia coli BL21 (DE3)/pRBS.

(2) Construction of a Strain Expressing Peptide Synthesizing Enzyme from Bacillus licheniformis ATCC14580 Strain

A DNA fragment having the nucleotide sequence shown by SEQ ID NO: 3 was obtained from the chromosomal DNA of the Bacillus licheniformis ATCC14580 strain as described below.

First, the Bacillus licheniformis ATCC14580 strain was spread on YPGA medium and then statically cultured overnight at 30° C. One platinum loop of the thus grown cells was inoculated in 3 mL of YPG medium, followed by 24 hours of shaking culture at 30° C. Cells were separated by centrifugation from the thus obtained culture solution and then chromosomal DNA was prepared using a Dneasy Kit from the thus obtained cells.

PCR was carried out using synthetic DNAs having the nucleotide sequences shown by SEQ ID NOS: 15 and 16 as a primer set and the chromosomal DNA of the Bacillus licheniformis ATCC14580 strain as a template. A reaction solution similar to that in (1) above was prepared and then PCR was carried out under reaction conditions similar to those in the same.

1/10 the amount of the reaction solution was subjected to agarose gel electrophoresis, so that the amplification by PCR of an approximately 1.2-kb DNA fragment was confirmed.

Next, the reaction solution was mixed with a TE-saturated phenol/chloroform (1 vol/1 vol) solution in an amount equivalent thereto. Cold ethanol was added in an amount twice that of an upper layer (obtained by centrifugation of the solution) to and mixed with the upper layer and then the solution was left to stand at −80° C. for 30 minutes. The solution was centrifuged to precipitate a DNA and then the DNA was dissolved in 20 μL of TE.

5 μL of the solution (in which the DNA had been dissolved) was subjected to digestion with restriction enzymes Nde I and BamH I, a DNA fragment was separated by agarose gel electrophoresis, and then an approximately 1.2-kb DNA fragment was collected using a Gene Clean II Kit.

After 0.2 μg of pET-21a(+) was subjected to digestion with restriction enzymes Nde_I and BamH I, a DNA fragment was separated by agarose gel electrophoresis, and then an approximately 5.4-kb DNA fragment was collected by a method similar to the above.

The thus obtained approximately 1.2-kb fragment and approximately 5.4-kb fragment were subjected to 16 hours of reaction at 16° C. for ligation using a ligation kit.

The Escherichia coli JM109 strain was transformed with the reaction solution by a method using calcium ions. The transformant was spread on LB agar medium containing 50 μg/mL ampicillin and then cultured overnight at 30° C., so that a transformed cell was selected.

The transformed cell was cultured overnight on LB medium containing 50 μg/ml ampicillin. A plasmid was prepared by an alkaline SDS method from the thus obtained culture solution.

Restriction enzyme digestion analysis was carried out. It was confirmed that the plasmid had a structure in which an approximately 1.2-kb DNA fragment (obtained above) had been inserted into pET-21a(+). This plasmid was designated pRBL.

Escherichia coli strain BL21 (DE3) was transformed with pRBL by a method using calcium ions. The transformant was spread on LB agar medium containing 50 μg/mL ampicillin and then cultured overnight at 30° C., so that a transformed cell was selected.

A plasmid was extracted by an alkaline SDS method from colonies of the transformant that had grown. The structure was analyzed using restriction enzymes, so as to confirm that pRBL was obtained.

The thus obtained transformed cell was designated Escherichia coli BL21 (DE3)/pRBL.

(3) Construction of a Strain Expressing Peptide Synthesizing Enzyme from Herpetosiphon aurantiacus ATCC23779 Strain

A DNA fragment having the nucleotide sequence shown by SEQ ID NO: 5 was obtained from the chromosomal DNA of the Herpetosiphon aurantiacus ATCC23779 strain as described below.

First, the Herpetosiphon aurantiacus ATCC23779 strain was spread on CY agar medium [3 g/L polypeptone (Nihon Pharmaceutical Co., Ltd.), 1 g/L yeast extract (Difco), 1 g/L calcium chloride, and 1.5 g/L agar] and then statically cultured overnight at 30° C. One platinum loop of the thus grown cells was inoculated in 3 mL of CY medium [3 g/L polypeptone (Nihon Pharmaceutical Co., Ltd.), 1 g/L yeast extract (Difco), 1 g/L calcium chloride], followed by 48 hours of shaking culture at 30° C. Cells were separated by centrifugation from the thus obtained culture solution and then chromosomal DNA was prepared using a Dneasy Kit from the thus obtained cells.

PCR was carried out using synthetic DNAs having the nucleotide sequences shown by SEQ ID NOS: 17 and 18 as a primer set and the chromosomal DNA of the Herpetosiphon aurantiacus ATCC23779 strain as a template. A reaction solution similar to that in (1) above was prepared and then PCR was carried out under reaction conditions similar to those in the same.

Next, 20 μL of a reaction solution containing 1.46 μL of the above reaction solution, 2.5 mmol/L dGTP, 5 mmol/L dithiothreitol (DTT), 1 unit of T4 DNA polymerase, and 2 μL of ×10 buffer for T4 DNA polymerase (Novagen) was prepared. After 30 minutes of reaction at 22° C., the reaction was stopped by heating at 75° C. for 20 minutes.

2 μL of the reaction solution was mixed with 1 μL of LIV vector pET-30 Xa/LIC (Novagen). After 5 minutes of reaction at 22° C., 1 μL of 25 mmol/L EDTA was added to the mixture and then a reaction was further carried out at 22° C. for minutes.

The Escherichia coli JM109 strain was transformed with the reaction solution by a method using calcium ions. The transformant was spread on LB agar medium containing 25 μg/mL kanamycin and then cultured overnight at 30° C., so that a transformed cell was selected.

The transformed cell was cultured overnight on LB medium containing 20 μg/ml kanamycin. A plasmid was prepared by an alkaline SDS method from the thus obtained culture solution.

Restriction enzyme digestion analysis was carried out. It was confirmed that the plasmid had a structure in which an approximately 1.2-kb DNA fragment (obtained above) had been inserted into pET-30 Xa/LIC. This plasmid was designated pRHA.

An Escherichia coli BL21 (DE3) strain was transformed with pRHA by a method using calcium ions. The transformant was spread on LB agar medium containing 50 μg/mL kanamycin and then cultured overnight at 30° C., so that a transformed cell was selected.

A plasmid was extracted by an alkaline SDS method from colonies of the transformant that had grown. The structure was analyzed using restriction enzymes, so as to confirm that pRHA was obtained.

The thus obtained transformed cell was designated Escherichia coli BL21 (DE3)/pRHA.

(4) Construction 1 of a Strain Expressing Peptide Synthesizing Enzyme from Streptococcus pneumoniae ATCC BAA-255 Strain

A DNA fragment having the nucleotide sequence shown by SEQ ID NO: 7 was obtained from the chromosomal DNA of the Streptococcus pneumoniae ATCC BAA-255 strain as described below.

The chromosomal DNA of Streptococcus pneumoniae ATCC BAA-255 strain was purchased from the American Type Culture Collection (ATCC).

PCR was carried out using synthetic DNAs having the nucleotide sequences shown by SEQ ID NOS: 19 and 20 as a primer set and the chromosomal DNA of the Streptococcus pneumoniae ATCC BAA-255 strain as a template. A reaction solution similar to that in (1) above was prepared and then PCR was carried out under reaction conditions similar to those in the same.

Next, 20 μL of a reaction solution containing 1.46 μL of the above reaction solution, 2.5 mmol/L dGTP, 5 mmol/L dithiothreitol (DTT), 1 unit of T4 DNA polymerase, and 2 μL of ×10 buffer for T4 DNA polymerase (Novagen) was prepared. After 30 minutes of reaction at 22° C., the reaction was stopped by heating at 75° C. for 20 minutes.

2 μL of the reaction, solution was mixed with 1 μL of LIV vector pET-30 Xa/LIC. After 5 minutes of reaction at 22° C., 1 μL of 25 mmol/L EDTA was added to the mixture and then a reaction was further carried out at 22° C. for minutes.

The Escherichia coli JM109 strain was transformed with the reaction solution by a method using calcium ions. The transformant was spread on LB agar medium containing 25 μg/mL kanamycin and then cultured overnight at 30° C., so that a transformed cell was selected.

The transformed cell was cultured overnight on LB medium containing 20 μg/ml kanamycin. A plasmid was prepared by an alkaline SDS method from the thus obtained culture solution.

Restriction enzyme digestion analysis was carried out. It was confirmed that the plasmid had a structure in which an approximately 1.2-kb DNA fragment (obtained above) had been inserted into pET-30 Xa/LIC. This plasmid was designated pRSP.

An Escherichia coli strain BL21 (DE3) was transformed with pRSP by a method using calcium ions. The transformant was spread on LB agar medium containing 25 μg/mL kanamycin and then cultured overnight at 30° C., so that a transformed cell was selected.

A plasmid was extracted by an alkaline SDS method from colonies of the transformant that had grown. The structure was analyzed using restriction enzymes, so as to confirm that pRSP was obtained.

The thus obtained transformed cell was designated Escherichia coli BL21 (DE3)/pRSP.

(5) Construction of a Strain Expressing Peptide Synthesizing Enzyme from Chromobacterium violaceum NBRC 12614 Strain

A DNA fragment having the nucleotide sequence shown by SEQ ID NO: 9 was obtained from the chromosomal DNA of the Chromobacterium violaceum NBRC12614 strain as described below.

First, the Chromobacterium violaceum NBRC12614 strain (available from Bioresource Information Center, Incorporated Administrative Agency National Institute of Technology and Evaluation, the NITE Biological Resource Center) was spread on NBRC medium 802 agar medium [10 g/L polypeptone (Nihon Pharmaceutical Co., Ltd.), 3 g/L yeast extract (Difco), 1 g/L magnesium sulfate, and 1.5 g/L agar] and then statically cultured at 30° C. overnight. One platinum loop of the thus grown cells was inoculated in 3 mL of NBRC medium 802 medium [10 g/L polypeptone (Nihon Pharmaceutical Co., Ltd.), 3 g/L yeast extract (Difco), and 1 g/L magnesium sulfate], followed by 24 hours of shaking culture at 30° C. Cells were separated by centrifugation from the thus obtained culture solution and then chromosomal DNA was prepared using a Dneasy Kit from the obtained cells.

PCR was carried out using synthetic DNAs having the nucleotide sequences shown by SEQ ID NOS: 21 and 22 as a primer set and the chromosomal DNA of the Chromobacterium violaceum NBRC12614 strain as a template. A reaction solution similar to that in (1) above was prepared and then PCR was carried out under reaction conditions similar to those in the same.

Next, 20 μL of a reaction solution containing 1.46 μL of the above reaction solution, 2.5 mmol/L dGTP, 5 mmol/L dithiothreitol (DTT), 1 unit of T4 DNA polymerase, and 2 μL of ×10 buffer for T4 DNA polymerase was prepared. After 30 minutes of reaction at 22° C., the reaction was stopped by heating at 75° C. for 20 minutes.

2 μL of the reaction solution was mixed with 1 μL of LIV vector pET-30 Xa/LIC. After 5 minutes of reaction at 22° C., 1 μL of 25 mmol/L EDTA was added to the mixture and then a reaction was further carried out at 22° C. for minutes.

The Escherichia coli JM109 strain was transformed with the reaction solution by a method using calcium ions. The transformant was spread on LB agar medium containing 25 μg/mL kanamycin and then cultured overnight at 30° C., so that a transformed cell was selected.

The transformed cell was cultured overnight on LB medium containing 20 μg/ml kanamycin. A plasmid was prepared by an alkaline SDS method from the thus obtained culture solution.

Restriction enzyme digestion analysis was carried out. It was confirmed that the plasmid had a structure in which an approximately 1.2-kb DNA fragment (obtained above) had been inserted into pET-30 Xa/LIC. This plasmid was designated pRCV.

An Escherichia coli Rosetta (DE3) strain (Novagen) was transformed with pRCV by a method using calcium ions. The transformant was spread on LB agar medium containing 25 μg/mL kanamycin and 25 μg/mL chloramphenicol and then cultured overnight at 30° C., so that a transformed cell was selected.

A plasmid was extracted by an alkaline SDS method from colonies of the transformant that had grown. The structure was analyzed using restriction enzymes, so as to confirm that pRCV was obtained.

The thus obtained transformed cell was designated Escherichia coli Rosetta (DE3)/pRCV.

(6) Construction of a Strain Expressing Peptide Synthesizing Enzyme from Bifidobacterium adolescentis JCM 1275 Strain

A DNA fragment having the nucleotide sequence shown by SEQ ID NO: 11 was obtained from the chromosomal DNA of the Bifidobacterium adolescentis JCM1275 strain as described below.

First, the Bifidobacterium adolescentis JCM 1275 strain (available from RIKEN BioResource Center, JAPAN COLLECTION OF MICROORGANISMS) was spread on GAM broth agar medium (Nissui Pharmaceutical Co., Ltd.) and then statically cultured in a sealed container using Anaero Pack Kenki (Mitsubishi Gas Chemical Company, Inc.) at 30° C. over two nights under anaerobic conditions. Colonies on plates were gathered after culturing, so that cells were collected. Chromosomal DNA was prepared using a Dneasy Kit from the obtained cells.

PCR was carried out using synthetic DNAs having the nucleotide sequences shown by SEQ ID NOS: 23 and 24 as a primer set and the chromosomal DNA of the Bifidobacterium adolescentis JCM1275 strain as a template. A reaction solution similar to that in (1) above was prepared and then PCR was carried out under reaction conditions similar to those in the same.

Next, 20 μL of a reaction solution containing 1.46 μL of the reaction solution, 2.5 mmol/L dGTP, 5 mmol/L dithiothreitol (DTT), 1 unit of T4 DNA polymerase, and 2 μL of ×10 buffer for T4 DNA polymerase was prepared. After 30 minutes of reaction at 22° C., the reaction was stopped by heating at 75° C. for 20 minutes.

2 μL of the reaction solution was mixed with 1 μL of LIV vector pET-30 Xa/LIC. After 5 minutes of reaction at 22° C., 1 μL of 25 mmol/L EDTA was added to the mixture and then a reaction was further carried out at 22° C. for minutes.

The Escherichia coli JM109 strain was transformed with the reaction solution by a method using calcium ions. The transformant was spread on LB agar medium containing 25 μg/mL kanamycin and then cultured overnight at 30° C., so that a transformed cell was selected.

The transformed cell was cultured overnight on LB medium containing 20 μg/ml kanamycin. A plasmid was prepared by an alkaline SDS method from the thus obtained culture solution.

Restriction enzyme digestion analysis was carried out. It was confirmed that the plasmid had a structure in which an approximately 1.2-kb DNA fragment (obtained above) had been inserted into pET-30 Xa/LIC. This plasmid was designated pRBAD.

Escherichia coli Rosetta (DE3) strain was transformed with pRBAD by a method using calcium ions. The transformant was spread on LB agar medium containing 25 μg/mL kanamycin and then cultured overnight at 30° C., so that a transformed cell was selected.

A plasmid was extracted by an alkaline SDS method from colonies of the transformant that had grown. The structure was analyzed using restriction enzymes, so as to confirm that pRBAD was obtained.

The thus obtained transformed cell was designated Escherichia coli BL21 (DE3)/pRBAD.

### Example 2

### Production of Proteins Having Peptide-Synthesizing Activity

Escherichia coli BL21 (DE3)/pRBS and Escherichia coli BL21 (DE3)/pRBL obtained in Example 1 (1) and (2) were inoculated in tubes each containing 3 mL of LB medium containing 50 μg/mL ampicillin, followed by 18 hours of shaking culture at 30° C. 1 mL of each culture solution obtained was introduced into a 500-mL Erlenmeyer flask containing 100 mL of LB medium containing 50 μg/mL ampicillin and 0.1 mmol/L isopropyl-β-D-thiogalactopyranoside (IPTG). After 24 hours of shaking culture at 25° C., the culture was centrifuged, so that wet cells were obtained.

Escherichia coli BL21 (DE3)/pRHA, Escherichia coli BL21 (DE3)/pRSP, and Escherichia coli BL21 (DE3)/pRBAD obtained in Example 1 (3), (4), and (6) were inoculated in tubes each containing 3 mL of LB medium containing 30 μg/mL kanamycin, followed by 5 hours of shaking culture at 37° C. 1 mL of each culture solution obtained was introduced into a 500-mL Erlenmeyer flask containing 100 mL of LB medium containing 30 μg/mL kanamycin and 0.4 mmol/L IPTG. After 19 hours of shaking culture at 30° C., the culture was centrifuged, so that wet cells were obtained.

Escherichia coli Rosetta (DE3)/pRCV obtained in Example 1 (5) was inoculated in a tube containing 3 mL of LB medium containing 30 μg/mL kanamycin and 30 μg/mL chloramphenicol, followed by 5 hours of shaking culture at 37° C. 1 mL of the thus obtained culture solution was introduced into a 500-mL Erlenmeyer flask containing 100 mL of LB medium containing 30 μg/mL kanamycin, 30 μg/mL chloramphenicol, and 0.4 mmol/L IPTG. After 19 hours of shaking culture at 30° C., the culture was centrifuged, so that wet cells were obtained.

The wet cells obtained above were disrupted by ultrasonication and then the resultants were each subjected to centrifugation. From the thus obtained supernatants, proteins having peptide-synthesizing activity were each purified using HisTrap (Amersham).

### Example 3

Production of Peptides from Single Amino Acids Using Proteins Having Peptide-Synthesizing Activity

Reaction solutions were prepared each containing purified protein (0.1 mg/mL) obtained in Example 2, 50 mmol/L Tris-HCl buffer (pH 8.0), 12.5 mmol/L magnesium sulfate, 12.5 mmol/L ATP, and one kind of L-amino acid (12.5 mmol/L) (selected from among L-arginine (L-Arg), L-alanine (L-Ala), L-glutamine (L-Gln), L-glutamic acid (L-Glu), L-valine (L-Val), L-leucine (L-Leu), L-isoleucine (L-Ile), L-proline (L-Pro), L-tyrosine (L-Tyr), L-phenylalanine (L-Phe), L-tryptophan (L-Trp), L-methionine (L-Met), L-serine (L-Ser), L-threonine (L-Thr), L-cysteine (L-Cys), L-asparagine (L-Asn), L-lysine (L-Lys), L-arginine (L-Arg), L-histidine (L-His), and L-aspartic acid (L-Asp)) or glycine (Gly). Peptide production reaction was carried out at 30° C. for 20 hours.

After completion of the reaction, the thus formed products were analyzed by three types of techniques including determination of the amounts of free phosphoric acid that had been formed in the reaction solutions by the peptide production reaction using a Determiner L IP (Kyowa Medex Co., Ltd.), LC/MS (Liquid Chromatography/Mass Spectrometry) analysis, and high-performance liquid chromatography (HPLC) analysis.

Table 1 to Table 6 show the results of reactions for which free phosphoric acid was detected by determination using a Determiner L IP. Table 1 to Table 6 show the results of reactions using peptide synthesizing enzyme produced in Example 2 using BL21 (DE3)/pRBS, BL21 (DE3)/pRBL, BL21 (DE3)/pRHA, BL21 (DE3)/pRSP, Rosetta (DE3)/pRCV, and BL21 (DE3)/pRBAD.

LC/MS analysis was conducted for samples for which free phosphoric acids had been detected using a Determiner L IP. Peptides detected by LC/MS analysis are shown in the right columns in Tables 1 to 6. In Tables 1 to 6, empty right columns indicate that peptides bound by α-peptide bonds were not detected by LC/MS analysis.

Furthermore, the concentrations of peptides, which could be measured by HPLC analysis, are shown in parentheses in the right columns in Tables 1 to 6.

Also, when Escherichia coli BL21 (DE3)/pRSP-derived protein was used and L-Val and L-Leu were used as substrates, it was confirmed that precipitates had been generated upon completion of a reaction carried out using BL21(DE3)/pRBAD and L-Trp and L-Phe as substrates. These precipitates were collected by centrifugation, a solution (acetic acid:water=1:1) was added thereto for dissolution, and then MALDI-TOFMS (Matrix Assisted Laser Desorption Ionization/Time Of Flight/Mass Spectrometry) analysis was conducted.

In addition, all amino acids composing peptides, the formed products, are L-amino acids, but this is not described in Tables for abbreviation.

TABLE 1
From Bacillus subtilis ATCC6633 strain

| Amino acid used as | | |
|---|---|---|
| substrate | Product | |
| L-Val | Supernatant | Val-Val (1.72 mM) |
| | | Val-Val-Val (2.68 mM) |
| | | Val-Val-Val-Val (0.12 mM) |
| | | Val-Val-Val-Val-Val |
| L-Leu | Supernatant | Leu-Leu |
| | | Leu-Leu-Leu |
| | | Leu-Leu-Leu-Leu |
| | | Leu-Leu-Leu-Leu-Leu |
| L-Ile | Supernatant | Ile-Ile |
| | | Ile-Ile-Ile |
| | | Ile-Ile-Ile-Ile |
| L-Met | Supernatant | Met-Met |
| | | Met-Met-Met |
| | | Met-Met-Met-Met |
| | | Met-Met-Met-Met-Met |
| L-Cys | Supernatant | |
| L-Lys | Supernatant | |
| L-Pro | Supernatant | |

TABLE 2
From Bacillus licheniformis ATCC14580 strain

| Amino acid used as | | |
|---|---|---|
| substrate | Product | |
| L-Val | Supernatant | Val-Val (1.15 mM) |
| | | Val-Val-Val (3.78 mM) |
| | | Val-Val-Val-Val (0.05 mM) |
| | | Val-Val-Val-Val-Val |
| L-Leu | Supernatant | Leu-Leu |
| | | Leu-Leu-Leu |
| | | Leu-Leu-Leu-Leu |
| | | Leu-Leu-Leu-Leu-Leu |
| L-Ile | Supernatant | Ile-Ile |
| | | Ile-Ile-Ile |
| | | Ile-Ile-Ile-Ile |
| L-Met | Supernatant | Met-Met |
| | | Met-Met-Met |
| | | Met-Met-Met-Met |
| | | Met-Met-Met-Met-Met |
| L-Cys | Supernatant | |
| L-Lys | Supernatant | |
| L-Pro | Supernatant | |

TABLE 3
From Herpetosiphon aurantiacus ATCC23779 strain

| | |
|---|---|
| Amino acid | |
| used as | |
| substrate | Product |

| | | |
|---|---|---|
| L-Val | Supernatant | Val-Val (2.12 mmol/L) |
| | | Val-Val-Val (1.46 mmol/L) |
| | | Val-Val-Val-Val (0.09 mmol/L) |
| L-Leu | Supernatant | Leu-Leu (0.13 mmol/L) |
| | | Leu-Leu-Leu (0.25 mmol/L) |
| | | Leu-Leu-Leu-Leu |
| L-Ile | Supernatant | Ile-Ile (0.22 mmol/L) |
| | | Ile-Ile-Ile |
| | | Ile-Ile-Ile-Ile |
| L-Met | Supernatant | Met-Met |
| | | Met-Met-Met |
| | | Met-Met-Met-Met |
| L-Cys | Supernatant | |
| L-Trp | Supernatant | Trp-Trp |
| L-Phe | Supernatant | Phe-Phe |
| | | Phe-Phe-Phe |

TABLE 4
From Streptococcus pneumoniae ATCC BAA-255 strain

| | |
|---|---|
| Amino acid | |
| used as | |
| substrate | Product |

| | | |
|---|---|---|
| L-Val | Supernatant | Val-Val (1.51 mmol/L) |
| | | Val-Val-Val (0.25 mmol/L) |
| | | Val-Val-Val-Val (0.67 mmol/L) |
| | | Val-Val-Val-Val-Val |
| | | Val-Val-Val-Val-Val-Val |
| | Precipitate | Val-Val-Val-Val-Val |
| | | Val-Val-Val-Val-Val-Val |
| L-Leu | Supernatant | Leu-Leu (0.22 mmol/L) |
| | | Leu-Leu-Leu (0.01 mmol/L) |
| | | Leu-Leu-Leu-Leu |
| | | Leu-Leu-Leu-Leu-Leu |
| | | Leu-Leu-Leu-Leu-Leu-Leu |
| | Precipitate | Leu-Leu-Leu-Leu-Leu |
| | | Leu-Leu-Leu-Leu-Leu-Leu |
| L-Ile | Supernatant | Ile-Ile (3.98 mmol/L) |
| | | Ile-Ile-Ile |
| | | Ile-Ile-Ile-Ile |
| | | Ile-Ile-Ile-Ile-Ile |
| | Precipitate | Ile-Ile-Ile-Ile |
| | | Ile-Ile-Ile-Ile-Ile |
| L-Met | Supernatant | Met-Met |
| | | Met-Met-Met |
| | | Met-Met-Met-Met |
| | | Met-Met-Met-Met-Met |
| | | Met-Met-Met-Met-Met-Met |
| L-Cys | Supernatant | |
| L-Trp | Supernatant | Trp-Trp |
| L-Phe | Supernatant | Phe-Phe |

TABLE 5
From Chromobacterium violaceum NBRC12614 strain

| | |
|---|---|
| Amino acid | |
| used as | |
| substrate | Product |

| | | |
|---|---|---|
| L-Val | Supernatant | Val-Val (0.54 mmol) |
| | | Val-Val-Val (1.59 mM) |
| | | Val-Val-Val-Val (0.86 mmol) |
| L-Leu | Supernatant | Leu-Leu |
| | | Leu-Leu-Leu |
| | | Leu-Leu-Leu-Leu |
| L-Ile | Supernatant | Ile-Ile |
| | | Ile-Ile-Ile |
| L-Met | Supernatant | Met-Met |
| | | Met-Met-Met |
| L-Cys | Supernatant | |
| L-Trp | Supernatant | Trp-Trp |
| L-Phe | Supernatant | |
| L-Ala | Supernatant | Ala-Ala |
| | | Ala-Ala-Ala |

TABLE 6
From Bifidobacterium adolescentis JCM1275 strain

| Amino acid | | |
|---|---|---|
| used as | | |
| substrate | Product | |
| L-Val | Supernatant | Val-Val (0.50 mmol/L) |
| | | Val-Val-Val (1.57 mmol/L) |
| | | Val-Val-Val-Val (0.04 mmol/L) |
| | | Val-Val-Val-Val-Val |
| L-Leu | Supernatant | Leu-Leu |
| | | Leu-Leu-Leu |
| | | Leu-Leu-Leu-Leu |
| | | Leu-Leu-Leu-Leu-Leu |
| | | Leu-Leu-Leu-Leu-Leu-Leu |
| L-Ile | Supernatant | Ile-Ile |
| | | Ile-Ile-Ile |
| | | Ile-Ile-Ile-Ile |
| | | Ile-Ile-Ile-Ile-Ile |
| L-Met | Supernatant | Met-Met |
| | | Met-Met-Met |
| | | Met-Met-Met-Met |
| | | Met-Met-Met-Met-Met |
| L-Cys | Supernatant | |
| L-Trp | Supernatant | Trp-Trp |
| | | Trp-Trp-Trp |
| | | Trp-Trp-Trp-Trp |
| | | Trp-Trp-Trp-Trp-Trp |
| | Precipitate | Trp-Trp-Trp |
| | | Trp-Trp-Trp-Trp |
| | | Trp-Trp-Trp-Trp-Trp |
| | | Trp-Trp-Trp-Trp-Trp-Trp |
| | | Trp-Trp-Trp-Trp-Trp-Trp-Trp |
| | | Trp-Trp-Trp-Trp-Trp-Trp-Trp-Trp |
| | | Trp-Trp-Trp-Trp-Trp-Trp-Trp-Trp-Trp |
| L-Phe | Supernatant | Phe-Phe |
| | | Phe-Phe-Phe |
| | | Phe-Phe-Phe-Phe |
| | | Phe-Phe-Phe-Phe-Phe |
| | Precipitate | Phe-Phe-Phe |
| | | Phe-Phe-Phe-Phe |
| | | Phe-Phe-Phe-Phe-Phe |
| | | Phe-Phe-Phe-Phe-Phe-Phe |
| L-Tyr | Supernatant | Tyr-Tyr |
| | | Tyr-Tyr-Tyr |
| | | Tyr-Tyr-Tyr-Tyr |
| | | Tyr-Tyr-Tyr-Tyr-Tyr |
| | | Tyr-Tyr-Tyr-Tyr-Tyr-Tyr |
| L-Asp | Supernatant | Asp-Asp |

As described above, it was revealed that the proteins of the present invention had activity of generating peptides in each of which 2 or more amino acids were bound via peptide bonds.

### Example 4

### Production of Heterologous Peptides Using Proteins Having Peptide-Synthesizing Activity

Peptide production reaction was carried out at 30° C. 20 hours using a reaction solution prepared comprising each peptide synthesizing enzyme (0.1 mg/mL) obtained in Example 2, 50 mmol/L Tris-HCl buffer (pH8.0), 12.5 mmol/L magnesium sulfate, 12.5 mmol/L ATP, and 2 kinds of amino acids or an amino acid derivative (12.5 mmol/L each) shown in Tables 7 to 12. Tables 7 to 12 show the results of each reaction using each peptide synthesizing enzyme produced in Example 2 using BL21(DE3)/pRBS, BL21(DE3)/pRBL, BL21(DE3)/pRHA, BL21(DE3)/pRSP, or BL21(DE3)/pRBAD. Also, in Tables 7 to 12, ArgHx denotes L-Arginine hydroxamate, ValOMe denotes L-Valine methyl ester, PheOMe denotes L-Phenylalaine methyl ester, and LeuOMe denotes L-Leucine methyl ester.

After completion of the reaction, phosphoric acids that had been liberated in the reaction solutions were determined using a Determiner L IP. As a result, liberation of phosphoric acid could be confirmed in all amino acid combinations shown in Tables 7 to 12.

All samples were further subjected to LC/MS analysis of formed products. The number of each amino acid residue contained in heterologous peptides formed in supernatants was estimated based on the molecular weights measured by LC/MS analysis. The results are shown in the right columns in Tables 7 to 12 (homologous peptides were also formed but are not described herein).

In addition, all amino acids composing peptides, the formed products, are L-amino acids, but this is not described in Tables for abbreviation.

TABLE 7
Peptide synthesizing enzyme from Bacillus subtilis ATCC6633 strain

| | | Produced heterologous peptide |
|---|---|---|
| | | (estimated number of amino |
| Substrate combination | | acid residues within peptides) |
| L-Val | L-Leu | (Val:Leu) = (2:1), (1:2), (3:1), (1:3), (2:2) |
| | L-Ile | (Val:Ile) = (2:1), (1:2), (3:1), (2:2) |
| | L-Met | (Val:Met) = (2:1), (1:2), (3:1), (1:3), (2:2) |
| | L-ArgHx | (Val:ArgHx) = (1:1), (2:1), (3:1) |
| | L-ValOMe | Val-ValOMe, Val-Val-ValOMe |
| | L-PheOMe | Val-PheOMe, Val-Val-PheOMe |
| L-Leu | L-Met | (Leu:Met) = (2:1), (1:2), (3:1), (2:2) |
| | L-ArgHx | Leu-ArgHx, Leu-Leu-ArgHx |
| L-Ile | L-Met | (Ile:Met) = (1:1), (2:1), (1:2), (1:3), (2:2) |

TABLE 8
Peptide synthesizing enzyme from Bacillus licheniformis
ATCC14580 strain

| | | Produced heterologous peptide |
|---|---|---|
| | | (estimated number of amino |
| Substrate combination | | acid residues within peptides) |
| L-Val | L-ArgHx | Val-ArgHx, Val-Val-ArgHx, Val-Val-Val-ArgHx |

TABLE 9
Peptide synthesizing enzyme from Herpetosiphon aurantiacus
ATCC23779 strain

| | |
|---|---|
| | Produced heterologous peptide |
| Substrate | (estimated number of amino acid |
| combination | residues within peptides) |

| | | |
|---|---|---|
| L-Val | L-Leu | (Val:Leu) = (1:1), (2:1), (1:2), |
| | | (3:1), (1:3), (2:2) |
| | L-Met | (Val:Met) = (1:1), (2:1), (1:2), |
| | | (3:1), (1:3) |
| | L-Arg | (Val:Arg) = (1:1), (2:1) |
| | L-ArgHx | Val-ArgHx, Val-Val-ArgHx |

TABLE 10
Peptide synthesizing enzyme from Streptococcus pneumoniae
ATCC BAA-255 strain

| | | Produced heterologous peptide |
|---|---|---|
| | | (estimated number of amino |
| Substrate combination | | acid residues within peptides) |
| L-Val | L-Arg | (Val:Arg) = (1:1) |
| | L-ArgHx | Val-ArgHx |
| | L-Tyr | (Val:Tyr) = (1:1) |
| | L-Pro | (Val:Pro) = (1:1), (2:1), (3:1), (4:1), (5:1) |
| | L-PheOMe | Val-PheOMe, Val-Val-PheOMe, |
| | | Val-Val-Val-PheOMe, |
| | | Val-Val-Val-Val-PheOMe |
| L-Leu | L-Tyr | (Leu:Tyr) = (1:1) |
| | L-Pro | (Leu:Pro) = (1:1), (2:1), (3:1), (4:1), (5:1) |
| | L-LeuOMe | Leu-LeuOMe, Leu-Leu-LeuOMe, |
| | | Leu-Leu-Leu-LeuOMe, |
| | | Leu-Leu-Leu-Leu-LeuOMe |
| L-Ile | L-Tyr | (Ile:Tyr) = (1:1) |
| | L-Pro | (Ile:Pro) = (1:1), (2:1), (3:1), (4:1) |

TABLE 11
Peptide synthesizing enzyme from Chromobacterium violaceum
NBRC12614 strain

| | |
|---|---|
| | Produced heterologous peptide |
| Substrate | (estimated number of amino |
| combination | acid residues within peptides) |

| | | |
|---|---|---|
| L-Val | L-Met | (Val:Met) = (1:1), (2:1), (3:1), (4:1) |
| | L-Tyr | (Val:Tyr) = (1:1) |
| | L-Ser | (Val:Ser) = (1:1), (2:1), (3:1) |
| | Gly | (Val:Gly) = (1:1), (2:1), (3:1) |
| | L-Ala | (Val:Ala) = (1:1), (2:1), (3:1) |
| | L-Asn | (Val:Asn) = (1:1), (2:1), (3:1) |

TABLE 12
Peptide synthesizing enzyme from Bifidobacterium adolescentis
JCM1275 strain

| | | Produced heterologous peptide |
|---|---|---|
| | | (estimated number of amino |
| Substrate combination | | acid residues within peptides) |
| L-Val | L-Tyr | (Val:Tyr) = (1:1), (2:1), (1:2), (1:3), (2:2) |
| | L-Trp | (Val:Trp) = (1:1), (2:1), (1:2), (1:3), (1:4) |
| | L-Phe | (Val:Phe) = (1:1), (2:1), (1:2), (1:3), (1:4) |
| L-Leu | L-Tyr | (Leu:Tyr) = (1:1), (2:1), (1:2), (3:1), (1:3), |
| | | (2:2), (4:1), (2:3), (3:2), (5:1), (4:2) |
| | L-PheOMe | Leu-PheOMe, Leu-Leu-PheOMe |
| | L-LeuOMe | Leu-LeuOMe, Leu-Leu-LeuOMe, |
| | | Leu-Leu-Leu-LeuOMe, |
| | | Leu-Leu-Leu-Leu-LeuOMe |
| L-Ile | L-Tyr | (Ile:Tyr) = (1:1), (2:1), (1:2), (3:1), (1:3), |
| | | (2:2), (1:4), (2:3), (3:2) |
| L-Tyr | L-Ser | (Tyr:Ser) = (2:1), (3:1) |
| | L-Thr | (Tyr:Thr) = (1:1), (2:1), (3:1), (4:1) |
| | L-Arg | (Tyr:Arg) = (1:1), (2:1) |
| | L-His | (Tyr:His) = (1:1), (2:1) |
| | L-Cys | (Tyr:Cys) = (1:1), (2:1), (3:1) |
| | L-Met | (Tyr:Met) = (1:1), (2:1), (1:2), (3:1), (1:3), |
| | | (2:2), (4:1), (1:4), (3:2), (2:3), (1:5) |

As described above, it was revealed that the proteins of the present invention had activity of generating various heterologous peptides in each of which 2 kinds of amino acids or amino acid derivatives were bound via peptide bonds.

### Example 5

Production (1) of Peptides Elongated from L-Amino Acids and Peptides Using Peptide Synthesizing Enzyme

Peptide production reaction was carried out at 30° C. 20 hours using a reaction solution prepared comprising each peptide synthesizing enzyme (0.1 mg/mL) obtained in Example 2, 50 mmol/L Tris-HCl buffer (pH8.0), 12.5 mmol/L magnesium sulfate, 12.5 mmol/L ATP, and a 12.5 mmol/L L-valine dipeptide (L-Val-L-Val) or a 12.5 mmol/L tripeptide (L-Val-L-Val-L-Val) and 12.5 mmol/L L-Val.

After completion of the reaction, formed products were analyzed by LC/MS analysis and HPLC analysis. The thus formed L-Val homologous peptides and concentrations thereof are shown in Tables 13 to 18.

In addition, all amino acids composing peptides, the formed products, are L-amino acids, but this is not described in Tables for abbreviation.

TABLE 13
Peptide synthesizing enzyme from Bacillus subtilis ATCC6633 strain

| L-amino | | | Concentration |
|---|---|---|---|
| acid | Peptide | Produced peptide | (mmol/L) |
| L-Val | L-Val-L-Val | Val-Val | 5.57 |
| | | Val-Val-Val | 7.36 |
| | | Val-Val-Val-Val | 0.19 |
| | L-Val-L-Val-L-Val | Val-Val | 0.68 |
| | | Val-Val-Val | 9.94 |
| | | Val-Val-Val-Val | 1.06 |

TABLE 14
Peptide synthesizing enzyme from Bacillus licheniformis
ATCC14580 strain

| L-amino | | | Concentration |
|---|---|---|---|
| acid | Peptide | Produced peptide | (mmol/L) |
| L-Val | L-Val-L-Val | Val-Val | 4.80 |
| | | Val-Val-Val | 8.76 |
| | | Val-Val-Val-Val | 0.05 |
| | L-Val-L-Val-L-Val | Val-Val | 0.47 |
| | | Val-Val-Val | 13.73 |
| | | Val-Val-Val-Val | 0.31 |

TABLE 15
Peptide synthesizing enzyme from Herpetosiphon aurantiacus
ATCC23779 strain

| L-amino | | | Concentration |
|---|---|---|---|
| acid | Peptide | Produced peptide | (mmol/L) |
| L-Val | L-Val-L-Val | Val-Val | 5.17 |
| | | Val-Val-Val | 3.75 |
| | | Val-Val-Val-Val | 0.08 |
| | L-Val-L-Val-L-Val | Val-Val | 0.93 |
| | | Val-Val-Val | 10.90 |
| | | Val-Val-Val-Val | 0.64 |

TABLE 16
Peptide synthesizing enzyme from Streptococcus pneumoniae
ATCC BAA-255 strain

| L-amino | | | Concentration |
|---|---|---|---|
| acid | Peptide | Produced peptide | (mmol/L) |
| L-Val | L-Val-L-Val | Val-Val | 6.79 |
| | | Val-Val-Val | 1.07 |
| | | Val-Val-Val-Val | 3.29 |
| | L-Val-L-Val-L-Val | Val-Val | 0.13 |
| | | Val-Val-Val | 2.49 |
| | | Val-Val-Val-Val | 6.77 |

TABLE 17
Peptide synthesizing enzyme from Chromobacterium violaceum
NBRC12614 strain

| L-amino | | | Concentration |
|---|---|---|---|
| acid | Peptide | Produced peptide | (mmol/L) |
| L-Val | L-Val-L-Val | Val-Val | 4.46 |
| | | Val-Val-Val | 7.32 |
| | | Val-Val-Val-Val | 1.20 |
| | L-Val-L-Val-L-Val | Val-Val | 0.17 |
| | | Val-Val-Val | 6.18 |
| | | Val-Val-Val-Val | 3.83 |

TABLE 18
Peptide synthesizing enzyme from Bifidobacterium adolescentis
JCM1275 strain

| L-amino | | | Concentration |
|---|---|---|---|
| acid | Peptide | Produced peptide | (mmol/L) |
| L-Val | L-Val-L-Val | Val-Val | 7.47 |
| | | Val-Val-Val | 5.85 |
| | | Val-Val-Val-Val | 0.04 |
| | L-Val-L-Val-L-Val | Val-Val | 0.22 |
| | | Val-Val-Val | 10.16 |
| | | Val-Val-Val-Val | 0.37 |

As described above, it was revealed that the proteins of the present invention had activity of forming peptides further elongated using peptides as substrates and peptide bonds.

### Example 6

Production (2) of Peptides Elongated from L-Amino Acids and Peptides Using Peptide Synthesizing Enzyme

Peptide production reaction was carried out at 30° C. for 20 hours using a reaction solution prepared comprising,0.1 mg/mL peptide synthesizing enzyme obtained from BL21(DE3)/pRSP, 50 mmol/L Tris-HCl buffer (pH8.0), 12.5 mmol/L magnesium sulfate, 12.5 mmol/L ATP, and a 12.5 mmol/L L-valine dipeptide (L-Val-L-Val) or a 12.5 mmol/L tripeptide (L-Val-L-Val-L-Val) and 12.5 mmol/L L-Pro.

After completion of the reaction, formed products were analyzed by LC/MS analysis. As a result, it was revealed that a peptide in which one molecule of L-Pro was bound to one molecule of L-Val-L-Val or L-Val-L-Val-L-Val was produced.

### Example 7

Production (3) of Peptides Elongated from L-Amino Acids and Peptides Using Peptide Synthesizing Enzyme

Peptide production reaction was carried out at 30° C. for 20 hours using a reaction solution prepared comprising 0.1 mg/mL peptide synthesizing enzyme obtained from BL21(DE3)/pRBS, 50 mmol/L Tris-HCl buffer (pH8.0), 12.5 mmol/L magnesium sulfate, 12.5 mmol/L ATP, 12.5 mmol/L L-arginyl-L-serine (L-Arg-L-Ser) or 12.5 mmol/L L-arginyl-L-glutamic acid (L-Arg-L-Glu) and 12.5 mmol/L L-Val. After completion of the reaction, formed products were analyzed by an LC/MS analysis method.

It was revealed that as a result of reaction using L-Val and L-Arg-L-Ser as substrates, a tripeptide was produced in which one molecule of L-Val was bound to one molecule of L-Arg-L-Ser and a tetrapeptide was produced in which two molecules of L-Val were bound to one molecule of L-Arg-L-Ser. It was also revealed that as a result of reaction using L-Val and L-Arg-L-Glu as substrates, a tripeptide was produced in which one molecule of L-Val was bound to one molecule of L-Arg-L-Glu and a tetrapeptide was produced in which two molecules of L-Val were bound to one molecule of L-Arg-L-Glu.

### Example 8

Construction 2 of a Strain Expressing Peptide Synthesizing Enzyme from Streptococcus pneumoniae ATCC BAA-255 Strain

A DNA fragment having the nucleotide sequence shown by SEQ ID NO: 7 was obtained from the chromosomal DNA of the Streptococcus pneumoniae ATCC BAA-255 strain as described below.

PCR was carried out using synthetic DNAs having the nucleotide sequences shown by SEQ ID NOS: 25 and 26 as a primer set and the chromosomal DNA of the Streptococcus pneumoniae ATCC BAA-255 strain as a template. Specifically, PCR was carried out by preparing a reaction solution similar to that in Example 1 (1) above under reaction conditions similar to those in the same.

Next, 20 μL of a reaction solution containing 1.46 μL of the above reaction solution, 2.5 mmol/L dGTP, 5 mmol/L dithiothreitol (DTT), 1 unit of T4 DNA polymerase, and 2 μL of ×10 buffer for T4 DNA polymerase was prepared and then reaction was carried out at 22° C. for 30 minutes. The reaction was stopped by heating at 75° C. for 20 minutes.

pTrcHis2B (Invitrogen Corporation) (0.2 μg) was digested with restriction enzymes BamH I and Hind III and then a DNA fragment was separated by agarose gel electrophoresis, so that an approximately 4.4-kb DNA fragment was collected by a method similar to the above method.

The thus obtained approximately 1.2-kb fragment and approximately 4.4-kb fragment were subjected to 16 hours of reaction at 16° C. for ligation using a ligation kit (Takara Bio Inc.).

The Escherichia coli JM109 strain was transformed with the reaction solution by a method using calcium ions. The transformant was spread on LB agar medium containing 50 μg/mL ampicillin and then cultured overnight at 30° C., so that a transformed cell was selected.

The transformed cell was cultured overnight on LB medium containing 50 μg/ml ampicillin. A plasmid was prepared by an alkaline SDS method from the thus obtained culture solution.

Restriction enzyme digestion analysis was carried out. It was confirmed that the plasmid had a structure in which an approximately 1.2-kb DNA fragment had been inserted into pTrcHis2B. This plasmid was designated pTrcSP.

Escherichia coli JPNDABPOPepT1 strain was transformed with pTrcSP by a method using calcium ions. The transformant was spread on LB agar medium containing 50 μg/mL ampicillin and then cultured overnight at 30° C., so that a transformed cell was selected.

A plasmid was extracted by an alkaline SDS method from colonies of the transformant that had grown. The structure was analyzed using restriction enzymes, so as to confirm that pTrcSP was obtained.

The thus obtained transformed cell was designated Escherichia coli JPNDABPOPepT1/pTrcSP.

### Example 9

### Production of a Val Homologous Peptide via Fermentation

Escherichia coli JPNDABPOPepT1/pTrcSP obtained in Example 8 was inoculated in tube containing 3 mL of LB medium containing 100 μg/mL ampicillin, followed by 5 hours of shaking culture at 37° C. Three (3) mL of the thus obtained culture solution was introduced into a 500-mL Erlenmeyer flask containing 100 ml of LB medium containing 100 μg/mL ampicillin, 0.4 mmol/L isopropyl-β-D-thiogalactopyranoside (IPTG), and 10 g/L L-valine, followed by 19 hours of shaking culture at 30° C. Subsequently, the total amount of the culture was centrifuged, so that supernatant of the culture and wet cells were obtained. For analysis of intracellular components, the thus obtained wet cells were suspended in 4 mL of 100 mmol/L Tris-HCl buffer (pH8.0). The suspension was ultrasonicated and then centrifuged to remove the residues. The thus obtained acellular extract was boiled (100° C., 5 min) and then centrifuged. The thus obtained supernatant was used as a sample for analysis of intracellular components.

Formed products were analyzed by LC/MS analysis. Specifically, the thus obtained supernatant of the culture at the initiation of culturing (immediately after inoculation) and the same at the completion of culturing (19 hours), as well as intracellular components at the completion of culturing, were analyzed.

As a result, the presence of Val homologous peptide was never confirmed in the supernatant of the culture at the initiation of culturing, but the presence of Val dimer, trimer, tetramer, and pentamer in the supernatant of the culture at the completion of culture was confirmed. Also, within cells at the completion of culture, the presence of Val dimer, trimer, tetramer, pentamer, and hexamer was confirmed.

### INDUSTRIAL APPLICABILITY

According to the present invention, a protein having peptide-synthesizing activity, a DNA encoding the protein, a recombinant DNA containing the DNA, a transformant obtained via transformation with the recombinant DNA, a process for producing a protein having peptide-synthesizing activity using the transformant and the like, a process for producing a peptide using a protein having peptide-synthesizing activity, and a process for producing a peptide using as an enzyme source a culture or the like of a transformant or a microorganism producing a protein having peptide-synthesizing activity are provided.

### Sequence Listing Free Text

SEQ ID NO: 13—explanation of artificial sequence: synthetic DNA
SEQ ID NO: 14—explanation of artificial sequence: synthetic DNA
SEQ ID NO: 15—explanation of artificial sequence: synthetic DNA
SEQ ID NO: 16—explanation of artificial sequence: synthetic DNA
SEQ ID NO: 17—explanation of artificial sequence: synthetic DNA
SEQ ID NO: 18—explanation of artificial sequence: synthetic DNA
SEQ ID NO: 19—explanation of artificial sequence: synthetic DNA
SEQ ID NO: 20—explanation of artificial sequence: synthetic DNA
SEQ ID NO: 21—explanation of artificial sequence: synthetic DNA
SEQ ID NO: 22—explanation of artificial sequence: synthetic DNA
SEQ ID NO: 23—explanation of artificial sequence: synthetic DNA
SEQ ID NO: 24—explanation of artificial sequence: synthetic DNA
SEQ ID NO: 25—explanation of artificial sequence: synthetic DNA
SEQ ID NO: 26—explanation of artificial sequence: synthetic DNA
SEQ ID NO: 27—explanation of artificial sequence: synthetic DNA
SEQ ID NO: 28—explanation of artificial sequence: synthetic DNA
SEQ ID NO: 29—explanation of artificial sequence: synthetic DNA
SEQ ID NO: 30—explanation of artificial sequence: synthetic DNA
SEQ ID NO: 31—explanation of artificial sequence: synthetic DNA
SEQ ID NO: 32—explanation of artificial sequence: synthetic DNA
SEQ ID NO: 33—explanation of artificial sequence: synthetic DNA
SEQ ID NO: 34—explanation of artificial sequence: synthetic DNA
SEQ ID NO: 35—explanation of artificial sequence: synthetic DNA
SEQ ID NO: 36—explanation of artificial sequence: synthetic DNA
SEQ ID NO: 37—explanation of artificial sequence: synthetic DNA
SEQ ID NO: 38—explanation of artificial sequence: synthetic DNA
SEQ ID NO: 39—explanation of artificial sequence: synthetic DNA
SEQ ID NO: 40—explanation of artificial sequence: synthetic DNA
SEQ ID NO: 41—explanation of artificial sequence: synthetic DNA
SEQ ID NO: 42—explanation of artificial sequence: synthetic DNA
SEQ ID NO: 43—explanation of artificial sequence: synthetic DNA
SEQ ID NO: 44—explanation of artificial sequence: synthetic DNA
SEQ ID NO: 45—explanation of artificial sequence: synthetic DNA
SEQ ID NO: 46—explanation of artificial sequence: synthetic DNA
SEQ ID NO: 47—explanation of artificial sequence: synthetic DNA
SEQ ID NO: 48—explanation of artificial sequence: synthetic DNA
SEQ ID NO: 49—explanation of artificial sequence: synthetic DNA
SEQ ID NO: 50—explanation of artificial sequence: synthetic DNA
SEQ ID NO: 51—explanation of artificial sequence: synthetic DNA
SEQ ID NO: 52—explanation of artificial sequence: synthetic DNA
SEQ ID NO: 53—explanation of artificial sequence: synthetic DNA
SEQ ID NO: 54—explanation of artificial sequence: synthetic DNA

1. A protein according to any one of the following [1] to [3]:
[1] a protein having the amino acid sequence shown by any one selected from the group consisting of SEQ ID NOS: 2, 4, 6, 8, 10, and 12;
[2] a protein consisting of an amino acid sequence that has a deletion, a substitution, or an addition of one or more amino acids in the amino acid sequence shown by any one selected from the group consisting of SEQ ID NOS: 2, 4, 6, 8, 10, and 12, and having peptide-synthesizing activity; and
[3] a protein consisting of an amino acid sequence that has 80% or more homology with the amino acid sequence shown by any one selected from the group consisting of SEQ ID NOS: 2, 4, 6, 8, 10, and 12, and having peptide-synthesizing activity.

2. A DNA according to any one of the following [1] to [3]:
[1] a DNA encoding the protein according to claim 1;
[2] a DNA having the nucleotide sequence shown by any one selected from the group consisting of SEQ ID NOS: 1, 3, 5, 7, 9, and 11; and
[3] a DNA hybridizing under stringent conditions to a DNA consisting of a nucleotide sequence complementary to the nucleotide sequence shown by any one selected from the group consisting of SEQ ID NOS: 1, 3, 5, 7, 9, and 11, and encoding a protein having peptide-synthesizing activity.

3. A recombinant DNA containing the DNA according to claim 2.

4. A transformant having the recombinant DNA according to claim 3.

5. The transformant according to claim 4, which is obtained using a microorganism as a host.

6. The transformant according to claim 5, wherein the microorganism belongs to the genus Escherichia.

7. A process for producing the protein of claim 1, which comprises culturing a microorganism which has an ability to produce the protein in a medium, allowing the protein to form and accumulate in a culture, and recovering the protein from the culture.

8. The process according to claim 7, wherein the microorganism belongs to the genus Bacillus, the genus Herpetosiphon, the genus Streptococcus, the genus Chromobacterium, or the genus Bifidobacterium.

9. The process according to claim 8, wherein the microorganism belonging to the genus Bacillus, the genus Herpetosiphon, the genus Streptococcus, the genus Chromobacterium, or the genus Bifidobacterium belongs to species selected from the group consisting of Bacillus subtilis, Bacillus lichenifonnis, Herpetosiphon aurantiacus, Streptococcus pneumoniae, Chromobacterium violaceum, and Bifidobacterium adolescentis.

10. The process according to claim 7, wherein the microorganism having an ability to produce the protein is a transformant containing a recombinant DNA according to any one of the following [1] to [3]:
[1] a DNA encoding the protein;
[2] a DNA having the nucleotide sequence shown by any one selected from the group consisting of SEQ ID NOS: 1, 3, 5, 7, 9, and 11; and
[3] a DNA hybridizing under stringent conditions to a DNA consisting of a nucleotide sequence complementary to the nucleotide sequence shown by any one selected from the group consisting of SEQ ID NOS: 1, 3, 5, 7, 9, and 11, and encoding a protein having peptide-synthesizing activity.

11. A process for producing a peptide, which comprises allowing the protein of claim 1, one or more kinds of substrates selected from among an amino acid, an amino acid derivative and a peptide, and adenosine-5′-triphosphate to be present in an aqueous medium, allowing the peptide to form and accumulate in the medium, and then recovering the peptide from the medium.

12. A process for producing a peptide, which comprises allowing a culture or a treated culture of a microorganism which has an ability to produce the protein of claim 1 and one or more kinds of substrates selected from among an amino acid, an amino acid derivative, and a peptide to be present in an aqueous medium, allowing the peptide to form and accumulate in the medium, and then recovering the peptide from the medium.

13. The process according to claim 12, wherein the microorganism which has an ability to produce the protein is a transformant containing a recombinant DNA according to any one of the following [1] to [3]:
[1] a DNA encoding the protein;
[2] a DNA having the nucleotide sequence shown by any one selected from the group consisting of SEQ ID NOS: 1, 3, 5, 7, 9, and 11; and
[3] a DNA hybridizing under stringent conditions to a DNA consisting of a nucleotide sequence complementary to the nucleotide sequence shown by any one selected from the group consisting of SEQ ID NOS: 1, 3, 5, 7, 9, and 11, and encoding a protein having peptide-synthesizing activity.

14. The process according to claim 12, wherein the microorganism which has an ability to produce the protein belongs to the genus Bacillus, the genus Herpetosiphon, the genus Streptococcus, the genus Chromobacterium, or the genus Bifidobacterium.

15. The process according to claim 14, wherein the microorganism belonging to the genus Bacillus, the genus Herpetosiphon, the genus Streptococcus, the genus Chromobacterium, or the genus Bifidobacterium belongs to species selected from the group consisting of Bacillus subtilis, Bacillus licheniformis, Herpetosiphon aurantiacus, Streptococcus pneumoniae, Chromobacterium violaceum, and Bifidobacterium adolescentis.

16. The process according to claim 12, wherein the treated culture is a concentrate of a culture, a dried culture, a cell obtained by centrifugation of a culture, a dried product of the cell, a freeze-dried product of the cell, the cell treated with a surfactant, an ultrasonicated product of the cell, a mechanically ground product of the cell, the cell treated with a solvent, the cell treated with an enzyme, a protein fraction of the cell, the cell in an immobilized form, or an enzymatic preparation obtained via extraction from the cell.

17. A process for producing a peptide, which comprises culturing a microorganism which has an ability to produce a protein having peptide-synthesizing activity and has an ability to produce at least one kind of substrate selected from the group consisting of an amino acid, an amino acid derivative, and a peptide in a medium, allowing the peptide to form and accumulate in the medium, and then recovering the peptide from the medium.

18. The process according to claim 17, wherein the protein having peptide-synthesizing activity is a protein according to any one of the following [1] to [3]:
[1] a protein having the amino acid sequence shown by any one selected from the group consisting of SEQ ID NOS: 2, 4, 6, 8, 10, and 12;
[2] a protein consisting of an amino acid sequence that has a deletion, a substitution, or an addition of one or more amino acids in the amino acid sequence shown by any one selected from the group consisting of SEQ ID NOS: 2, 4, 6, 8, 10, and 12, and having peptide-synthesizing activity; and
[3] a protein consisting of an amino acid sequence that has 80% or more homology with the amino acid sequence shown by any one selected from the group consisting of SEQ ID NOS: 2, 4, 6, 8, 10, and 12, and having peptide-synthesizing activity.

19. The process according to claim 18, wherein the microorganism which has an ability to produce the protein has an ability to produce at least one kind of substrate selected from the group consisting of an amino acid, an amino acid derivative, and a peptide is a transformant containing a recombinant DNA according to any one of the following [1] to [1]:
[1] a DNA encoding the protein;
[2] a DNA having the nucleotide sequence shown by any one selected from the group consisting of SEQ ID NOS: 1, 3, 5, 7, 9, and 11; and
[3] a DNA hybridizing under stringent conditions to a DNA consisting of a nucleotide sequence complementary to the nucleotide sequence shown by any one selected from the group consisting of SEQ ID NOS: 1, 3, 5, 7, 9, and 11, and encoding a protein having peptide-synthesizing activity.


SEQUENCE LISTING (54 sequences)

SEQ ID NO: 1 - DNA, 1227 bp, Bacillus subtilis
  CDS (1)..(1227)
attagcattttaatactgaacaaaacctcatattctaaatctccgtatgatttatggctaaaggatttagaagaacctgttgtaatgctgacttcaactgaacggttgcacgaatgcaaaaactatgatcagatcgagtcatttgatgagtatccggttaatggatgtattgaaatcagagcgctggaattacatgaaacatatagttttaacaccataattgcaatgtctgagtatgatcttttaagagcaggtaaacttcgtactcatttaggattgaaagggcagtcttatgagagtgcactattgtttcgggacaaagttttaatgaaacagcgtttggaagaacaggggattcctgttccccattaccggaaaattgaatccccggttgacctgtacctttttgtacagcagtttggttttccggtagtagtcaagccaatagatgggtcgggatctgtcgatacaaaggtgctaaaaaatgaaaaagacatgatgaaatacttatctaagggtcttgatggaaatgttgaagtggaaacgtttgtagatggtgatatgtaccatattgatgggcttatgatagacggacatattacattaaattggccctctcggtatataaacggatgtttggctttccaagaggaacaatttttagccagttatcagcttggcgctgacaacccattgacctccagattaattggcattgttgaaaaagcactaaaggcgctgccatccccaaaaacaactacttttcacgcggaagtgtttcatactccggatgataagctagtgttttgtgagattgctagccggactggaggggggttaatacgtgaggctattcagcaaggatttggttttgacttgaatgaggtttgtgtgaaaaaacagtgcggcctgccctatgatattccagattattcacagcttaaaatagggccaaaacagttaggcgggtggattttgatccctcctaaatacgggcggttggtcaaaattccttcaattccttttgagaattgggtaacaaagcaaaaagtgtctgctagagaaggagaggtgtttcagggtgcttcttctagtgtagacgttgtatcaagttacttaattaaagggaattcggaagatgtactgatcaatagaattcatcaagttgcctcatggtgcagtgaaaatatgatttgggaaggcaaatcaacgtgttctctcaaataa

SEQ ID NO: 2 - protein, 408 aa, Bacillus subtilis
ISILILNKTSYSKSPYDLWLKDLEEPVVMLTSTERLHECKNYDQIESFDEYPVNGCIEIRALELHETYSFNTIIAMSEYDLLRAGKLRTHLGLKGQSYESALLFRDKVLMKQRLEEQGIPVPHYRKIESPVDLYLFVQQFGFPVVVKPIDGSGSVDTKVLKNEKDMMKYLSKGLDGNVEVETFVDGDMYHIDGLMIDGHITLNWPSRYINGCLAFQEEQFLASYQLGADNPLTSRLIGIVEKALKALPSPKTTTFHAEVFHTPDDKLVFCEIASRTGGGLIREAIQQGFGFDLNEVCVKKQCGLPYDIPDYSQLKIGPKQLGGWILIPPKYGRLVKIPSIPFENWVTKQKVSAREGEVFQGASSSVDVVSSYLIKGNSEDVLINRIHQVASWCSENMIWEGKSTCSLK

SEQ ID NO: 3 - DNA, 1218 bp, Bacillus licheniformis
  CDS (1)..(1218)
atgagtacgcttatcttaaataaaacagcttatagcaagtctccgtatgatgtatggctgaagggactggatgaaccggtggcgatgctgacctcaaccgatcgtgcggatgaatgcaggaattatgatgtgatcgaagcatttgacgattatccggttaatggatgcatcgaaatgcgcgctttggagctgaatgaaacgtatcactttaaaacgattattgcatcctcggaatttgatatgctgagggcggggaagctgaggtcgcttttggggataaaagggcagtcttacaaaagtgctctgctgttccgcaacaaaatattaatgaaagagcaagtaagaaaagccggtgtgaaggttccagattttaagaaaatcgactcggcggccgacttatatacgtttgttcaaactttcggttttcctgtggtcgttaagcctatttacggatcggggtctgttgatacaaccgttttaaggaatcggcaggacgtgtggaatgttcttgcgaaaggtcttcccgatcatctggaagttgagtcgtttatcgaaggggatatgtatcatatagacggcctcatccttgatgaagaaattgtcctcagctggccgtcgcgttatatcaacgggtgtctagcgtttcaagatcatcagtttttaggcagcctccagcttgaattgaaaaatccgcttacccggcggttgactgattttgttcaaaaagcgcttgctgcccttcctgttccgaataccacaacattccatgctgaagtgtttcatacaccagatgatgaactggtgttttgtgaagtggccagtagaaccggcggcgctatggtccgtgaagcaactgtgcagactttcggttttgatatcaatgaagtgtgtgttaaagcgcaatgcggcctggaatttcatataccggaggtggacaacaatccggaccgcctaagcggctggctgttgattccgccgaaagacggcgtgctgaaagaaatcaagcctgcgccagccgcagactggatagcgaaacaaaatatatccgctcaagccggagacaggttccacggctcgtcttccagtgtagacgcaattgcaagctgtttaattatcggacgcacagaagccgaattgagaggccgtatcagccggcttgcaacgtggttccatgatcataccgtatgggaaaagaattcggatgtttgcgcaatttaa

SEQ ID NO: 4 - protein, 405 aa, Bacillus licheniformis
MSTLILNKTAYSKSPYDVWLKGLDEPVAMLTSTDRADECRNYDVIEAFDDYPVNGCIEMRALELNETYHFKTIIASSEFDMLRAGKLRSLLGIKGQSYKSALLFRNKILMKEQVRKAGVKVPDFKKIDSAADLYTFVQTFGFPVVVKPIYGSGSVDTTVLRNRQDVWNVLAKGLPDHLEVESFIEGDMYHIDGLILDEEIVLSWPSRYINGCLAFQDHQFLGSLQLELKNPLTRRLTDFVQKALAALPVPNTTTFHAEVFHTPDDELVFCEVASRTGGAMVREATVQTFGFDINEVCVKAQCGLEFHIPEVDNNPDRLSGWLLIPPKDGVLKEIKPAPAADWIAKQNISAQAGDRFHGSSSSVDAIASCLIIGRTEAELRGRISRLATWFHDHTVWEKNSDVCAI

SEQ ID NO: 5 - DNA, 1227 bp, Herpetosiphon aurantiacus
  CDS (1)..(1227)
atgaagatcttagtgctcaatcgtcaaaagcctcacttagctccgtttggcgattggcttggcgatttagtgccacaggcccgcttatttacggctgccaaccgtgtgcagggctttcaagggtttgcggcgattcagccatttgagaactatgaagacagtggcctgattgaatttgaggctttacggctgcatcgtcaatcgccaatcgagcgaattgttgcaacttcagaggtcgatattctgcgtgcaggccgcttacgtagctatcttgggttgccaggccaacaagccgatagtgccttggcctttcgcaataaagttgtgatgaagcaacacctggttaatcgcactcagctggtcaatatcccaatctttcaggcgatcaacgagccgttcgatatcattcaatttatcgaacagcatggctacccagtaatcgtcaaaccagatgatggcagtggctcgctgggggcaaaaatgctggcaaacgaggatgatctggcccagtttttacaacagccgctgccccgtggtttagaaattgagtgctttatccaaggcgatcaatatcatgtcgatggattattggtcgataacgaggtctgtttctgctggccatcgcaatatcttggcaatggtttatcctttacccaaggctggtttactgcgagccagatgcttcggcccgaacatcacttgacccagcgcttaatcgcagcggccaaagaagtgttggctttgctgccaactccacccgtcaccagctttcaccttgagttgtttcatactcctggcgatgagctgttcttttgcgaaattgccagccgcactggtggcggtatgatcaacggaacaattgagcaggcatttgggattaatctaaatcaactctttatccaaggccaagctggcatgccgattgatacgagccgattaagggcgatcacccaacccaagaagattgtcggttgggggttggttccaccgcaagctggggtttttcgcggctatcgccaagcaaaaccaccccaaccatgggtcctccacttcgattggagcattcaggcaggtacgcactcacaaccagcgcaaatgagtgttgatcaagtcggcgggtttattgttgatctgactgatgctcctaaccccgaagaacgcttgattgaggtttggcgctgggccgagcaccaagcactgtgggagccagcaggagtaaatgcatga

SEQ ID NO: 6 - protein, 408 aa, Herpetosiphon aurantiacus
MKILVLNRQKPHLAPFGDWLGDLVPQARLFTAANRVQGFQGFAAIQPFENYEDSGLIEFEALRLHRQSPIERIVATSEVDILRAGRLRSYLGLPGQQADSALAFRNKVVMKQHLVNRTQLVNIPIFQAINEPFDIIQFIEQHGYPVIVKPDDGSGSLGAKMLANEDDLAQFLQQPLPRGLEIECFIQGDQYHVDGLLVDNEVCFCWPSQYLGNGLSFTQGWFTASQMLRPEHHLTQRLIAAAKEVLALLPTPPVTSFHLELFHTPGDELFFCEIASRTGGGMINGTIEQAFGINLNQLFIQGQAGMPIDTSRLRAITQPKKIVGWGLVPPQAGVFRGYRQAKPPQPWVLHFDWSIQAGTHSQPAQMSVDQVGGFIVDLTDAPNPEERLIEVWRWAEHQALWEPAGVNA

SEQ ID NO: 7 - DNA, 1200 bp, Streptococcus pneumoniae
  CDS (1)..(1200)
atggtcaaaatagcaatcatcaatcaattttcacctagagtttgtgattataaagcgattaaagaattagaagataaaaaatatgaaattgaaattttcacaaaagctagatttaaatcttactatgaggattcaaagtttaaatgttatttctatgataatcttttggataaccaaaattatatatttgatattatagattcacattcaaaaaaaccatttacctacatagttgcaactcatgaatttgatatagttttggctgcaaagataagaaagttattgggaatatctggacaaaacattgatagtgctaatggctttagagataaatatataatgaaaaaatatttagaaaaagcagtatcgttaccaaaatatgctgaaattaatgattgtgtagatttaattgaatttactgataacaaaaaatatccatttgtagtgaaacctagacttggtgcaggatcaattggtgtaacaataatacaaaataaatctgaattaaaagattttattagtagtcctctaagtcaaaatttaatggttgaaactttcacaaatggggagatgtaccatgtggatggtttatttaaagataatgaaatgctgttatattctgtttcaaaatatttcaatgattgtttatcgtttaaaactaatacaccgctaggctcatatatgattgatgatagcaatcccctttcgagaaaattatatgatgcaacattaaaggtattaactaaaataccgactcctaatcatacaattccttttcatgctgaattttttgtagatggtgagaaggttactttttgcgaaattgcttctagagtaggaggagggctgataaatgattccttcaaattactaactaatattgatttacaaaaaacattcgtaaaaagtcaaataggaattgattatttaaatataataaaatctgataagagaacagcttggataacaattcctcctaaagaaggagaattactatctgttaatttgtataaagattcatgggttgagaaagtgaattttgatgagacctctattgggagaagatatagtggcggtgattatagcgcttctgctcttatagcatatttgattagtggaactgatgaagacgatttgaagaataaaattttacatattattgagtggcaagataaaaatacaatatataaagagaaataa

SEQ ID NO: 8 - protein, 399 aa, Streptococcus pneumoniae
MVKIAIINQFSPRVCDYKAIKELEDKKYEIEIFTKARFKSYYEDSKFKCYFYDNLLDNQNYIFDIIDSHSKKPFTYIVATHEFDIVLAAKIRKLLGISGQNIDSANGFRDKYIMKKYLEKAVSLPKYAEINDCVDLIEFTDNKKYPFVVKPRLGAGSIGVTIIQNKSELKDFISSPLSQNLMVETFTNGEMYHVDGLFKDNEMLLYSVSKYFNDCLSFKTNTPLGSYMIDDSNPLSRKLYDATLKVLTKIPTPNHTIPFHAEFFVDGEKVTFCEIASRVGGGLINDSFKLLTNIDLQKTFVKSQIGIDYLNIIKSDKRTAWITIPPKEGELLSVNLYKDSWVEKVNFDETSIGRRYSGGDYSASALIAYLISGTDEDDLKNKILHIIEWQDKNTIYKEK

SEQ ID NO: 9 - DNA, 1179 bp, Chromobacterium violaceum
  CDS (1)..(1179)
atgaagatcctgatcattcaccaagtgccttatcccaagatgcagtaccacttggggctggatcatcagcagcatgacatcacctatatcggctatccggcacggatggcggatcttcccgcctcattgcgggcgcaacgcctgttattgaatgacggcgaggatttggccgacggcgtgatccggcagacttcgccgcaggatggttaccaggcggtgttgtctttgtcggagttcggcattctgcaggcctaccgggtccgacagcatctgggcgtgccgggcgatgacctggcgatgttgcagcgcgtgcgcgacaaggtggcaatgaaggcggcgctgcatggctcgggcatcgatttcccgcgttttttccccgcccaggggctgagcgcgccagactggcagggccaaaccgtgctcaagccccggaaaggcgcctccagcgagggcgtcaggatctacccggcaatggaccaggcctgggccgcgtttgctgagctgcaggatggcgaggactggcagctggaagaatacattgaaggcagcatccatcatgccgatggcttggtgcaagccggccagctggtcgatctgaccgtcagccgctatctgaacaagcccgtcgatttcgccgagggcaacccgctcggttcctaccaattgcccactgatccgcgtcatttcgccttcgctgtggacgtggtgcaggccctgggcatacgtgacggttgcttgcacctggagttttttgaaaccgccgataagcggctggtgttcctggaagtggcaaaccggatggggggcgccggcgtggtcgacgcgcattggcgccatggcggcatccatttgcccagccacgaaatcgccatccgcctagggttgcccaggccgcggccgcagccaggcagcgggcgtttccacggttggctggttttccccggccatcatctgggggaaggcgaagccgacctgcgcttgccggaagacttggccagcgatccccgcgtggaccgtttgcatacgctgccggccgggcaggcgctcagccagcacatcacgtatcacgagtggcaggtgccggttttcatcgaagcttcggatgaaagcccgcaggccttggcggtttttttcaacgattgcatgcgacgtatccaaataaacaggaggccgatatga

SEQ ID NO: 10 - protein, 392 aa, Chromobacterium violaceum
MKILIIHQVPYPKMQYHLGLDHQQHDITYIGYPARMADLPASLRAQRLLLNDGEDLADGVIRQTSPQDGYQAVLSLSEFGILQAYRVRQHLGVPGDDLAMLQRVRDKVAMKAALHGSGIDFPRFFPAQGLSAPDWQGQTVLKPRKGASSEGVRIYPAMDQAWAAFAELQDGEDWQLEEYIEGSIHHADGLVQAGQLVDLTVSRYLNKPVDFAEGNPLGSYQLPTDPRHFAFAVDVVQALGIRDGCLHLEFFETADKRLVFLEVANRMGGAGVVDAHWRHGGIHLPSHEIAIRLGLPRPRPQPGSGRFHGWLVFPGHHLGEGEADLRLPEDLASDPRVDRLHTLPAGQALSQHITYHEWQVPVFIEASDESPQALAVFFNDCMRRIQINRRPI

SEQ ID NO: 11 - DNA, 1188 bp, Bifidobacterium adolescentis
  CDS (1)..(1188)
atgaaagtattattgctgcaacagcccaaatctttctcaaattatcctaaatggattgaggaaatccaagaacgtttcgattgtttagaagttatggtcttcacctcgaacgatcgagccgcacaccatagttggccgagctccgtcatcaaggaaatcgaggtttccgattattcctctgatagcgctaccgcaaaattctttgatattgtcaggaaatttaaaccagataggatagtttccagctcagaggaagacgtactgagggttgcagaggcgagaagtttatttggaattcccggtttacagcatgaactggcgttaagctgtcgagataaagtcacgatgaaacaatcggcccttgatgccggattaaagattattccctataccacctgccaaggctttggagatattatttcggcgttcgatcgttgggagacggttgttctcaaacctcgatggggcgcagggtctgcaggcattacgatattgcattcgaaagatgacctgcctgctttagctaccaagccggaattcatacgaaacgtgcattcaaaccagtattacttagaggaatattgttccggttccgtctaccatgtggatgtagtctatatcaattccggttcgattctgatctcaccatcacggtaccttgtcccgccactggatttcgagaaacaaaacaccggttccgtgatgttggatgagaacggtgccgactattccgagctgctccgactgacaaagcaattaattgcatctttcaacgatcagacgattccgaatgtgatgcatatcgaattctataagaatgaaaccggtgatttcgtattcggggaaatggcggcacgcagaggaggcggtctcatcaagcaggagctggcagccgcctatggcatagaccaaagcaaggccaacttcctattggaacttggtcttgtcgatgcggatgcgaatattacgcgatcatctcagtatggcatactgctggaaaccgctggattgaactggccaaaggaaaaagaaatcccggattgggcggttttggaatctgtgggcaagaaaaaaggtatcgctcataactctgttgattctgacaggaaatttcttatttccggcaagaatgaatctgagattattcaacgttctaattatcttataaatagtcaatataatgagtaa

SEQ ID NO: 12 - protein, 395 aa, Bifidobacterium adolescentis
MKVLLLQQPKSFSNYPKWIEEIQERFDCLEVMVFTSNDRAAHHSWPSSVIKEIEVSDYSSDSATAKFFDIVRKFKPDRIVSSSEEDVLRVAEARSLFGIPGLQHELALSCRDKVTMKQSALDAGLKIIPYTTCQGFGDIISAFDRWETVVLKPRWGAGSAGITILHSKDDLPALATKPEFIRNVHSNQYYLEEYCSGSVYHVDVVYINSGSILISPSRYLVPPLDFEKQNTGSVMLDENGADYSELLRLTKQLIASFNDQTIPNVMHIEFYKNETGDFVFGEMAARRGGGLIKQELAAAYGIDQSKANFLLELGLVDADANITRSSQYGILLETAGLNWPKEKEIPDWAVLESVGKKKGIAHNSVDSDRKFLISGKNESEIIQRSNYLINSQYNE

SEQ ID NO: 13 - DNA, 38 bp, Artificial
  Synthetic DNA
ggaattccatatgagcattttaatactgaacaaaacct

SEQ ID NO: 14 - DNA, 29 bp, Artificial
  Synthetic DNA
cgcggatccgatttgagagaacacgttga

SEQ ID NO: 15 - DNA, 32 bp, Artificial
  Synthetic DNA
gggaattccatatgagtacgcttatcttaaat

SEQ ID NO: 16 - DNA, 26 bp, Artificial
  Synthetic DNA
cgggatccgaaattgcgcaaacatcc

SEQ ID NO: 17 - DNA, 38 bp, Artificial
  Synthetic DNA
ggtattgagggtcgcatgaagatcttagtgctcaatcg

SEQ ID NO: 18 - DNA, 37 bp, Artificial
  Synthetic DNA
agaggagagttagagcctcatgcatttactcctgctg

SEQ ID NO: 19 - DNA, 40 bp, Artificial
  Synthetic DNA
ggtattgagggtcgcatggtcaaaatagcaatcatcaatc

SEQ ID NO: 20 - DNA, 51 bp, Artificial
  Synthetic DNA
agaggagagttagagccttatttctctttatatattgtatttttatcttgc

SEQ ID NO: 21 - DNA, 37 bp, Artificial
  Synthetic DNA
ggtattgagggtcgcatgaagatcctgatcattcacc

SEQ ID NO: 22 - DNA, 36 bp, Artificial
  Synthetic DNA
agaggagagttagagcctcatatcggcctcctgttt

SEQ ID NO: 23 - DNA, 39 bp, Artificial
  Synthetic DNA
ggtattgagggtcgcatgaaagtattattgctgcaacag

SEQ ID NO: 24 - DNA, 52 bp, Artificial
  Synthetic DNA
agaggagagttagagccttactcattatattgactatttataagataattag

SEQ ID NO: 25 - DNA, 34 bp, Artificial
  Synthetic DNA
cgggatccgatggtcaaaatagcaatcatcaatc

SEQ ID NO: 26 - DNA, 42 bp, Artificial
  Synthetic DNA
cgaagcttttatttctctttatatattgtatttttatcttgc

SEQ ID NO: 27 - DNA, 56 bp, Artificial
  Synthetic DNA
ctaaccctgtgacctgcaatactgttttgcgggtgagtgtaggctggagctgcttc

SEQ ID NO: 28 - DNA, 56 bp, Artificial
  Synthetic DNA
gaaactgccggaaggcgattaaacgccatccggcagcatatgaatatcctccttag

SEQ ID NO: 29 - DNA, 56 bp, Artificial
  Synthetic DNA
ttacgcaacaggaatagactgaacaccagactctatgtgtaggctggagctgcttc

SEQ ID NO: 30 - DNA, 56 bp, Artificial
  Synthetic DNA
agaaaacaggggtaaattccccgaatggcggcgctacatatgaatatcctccttag

SEQ ID NO: 31 - DNA, 56 bp, Artificial
  Synthetic DNA
atggagtttagtgtaaaaagcggtagcccggagaaagtgtaggctggagctgcttc

SEQ ID NO: 32 - DNA, 56 bp, Artificial
  Synthetic DNA
ttactcttcgccgttaaacccagcgcggtttaacagcatatgaatatcctccttag

SEQ ID NO: 33 - DNA, 56 bp, Artificial
  Synthetic DNA
atgacagaagcgatgaagattaccctctctacccaagtgtaggctggagctgcttc

SEQ ID NO: 34 - DNA, 56 bp, Artificial
  Synthetic DNA
ttacgccgttaacagattagctatcgtgcgcacacccatatgaatatcctccttag

SEQ ID NO: 35 - DNA, 56 bp, Artificial
  Synthetic DNA
cgccaatctttgttgtgaattactggcttagcttaagtgtaggctggagctgcttc

SEQ ID NO: 36 - DNA, 56 bp, Artificial
  Synthetic DNA
ttcgaagggtaagtttggatttaagccggatgcggccatatgaatatcctccttag

SEQ ID NO: 37 - DNA, 56 bp, Artificial
  Synthetic DNA
gcatccccacctcataacgttgacccgaccgggcaagtgtaggctggagctgcttc

SEQ ID NO: 38 - DNA, 56 bp, Artificial
  Synthetic DNA
ctgtacggcattttgctatgcttgtcgccactgttgcatatgaatatcctccttag

SEQ ID NO: 39 - DNA, 56 bp, Artificial
  Synthetic DNA
atgctggttaataccagtaattataatgagggagtcgtgtaggctggagctgcttc

SEQ ID NO: 40 - DNA, 56 bp, Artificial
  Synthetic DNA
tatcatctggcgggaataaataatcgcttttagcgtcatatgaatatcctccttag

SEQ ID NO: 41 - DNA, 21 bp, Artificial
  Synthetic DNA
gtgtctgaactgtctcaatta

SEQ ID NO: 42 - DNA, 21 bp, Artificial
  Synthetic DNA
cggaatttctttcagcagttc

SEQ ID NO: 43 - DNA, 21 bp, Artificial
  Synthetic DNA
atgactcaacagccacaagcc

SEQ ID NO: 44 - DNA, 21 bp, Artificial
  Synthetic DNA
tgctttagttatcttctcgta

SEQ ID NO: 45 - DNA, 21 bp, Artificial
  Synthetic DNA
agtgcctgcatcgtcgtgggc

SEQ ID NO: 46 - DNA, 21 bp, Artificial
  Synthetic DNA
ggcgccttttgctttaccaga

SEQ ID NO: 47 - DNA, 21 bp, Artificial
  Synthetic DNA
gacgcgcgctggggagaaaaa

SEQ ID NO: 48 - DNA, 21 bp, Artificial
  Synthetic DNA
cgtagcgcccgcagaccactg

SEQ ID NO: 49 - DNA, 24 bp, Artificial
  Synthetic DNA
tacgtgtctctggatacccaatca

SEQ ID NO: 50 - DNA, 24 bp, Artificial
  Synthetic DNA
atagttgtaaccgccagtgaacag

SEQ ID NO: 51 - DNA, 21 bp, Artificial
  Synthetic DNA
atgcgtatttccttgaaaaag

SEQ ID NO: 52 - DNA, 21 bp, Artificial
  Synthetic DNA
ttattcgatagagacgttttc

SEQ ID NO: 53 - DNA, 24 bp, Artificial
  Synthetic DNA
atgaccaacatcaccaagagaagt

SEQ ID NO: 54 - DNA, 24 bp, Artificial
  Synthetic DNA
ttagtgcttcacaatgtacatatt